(19) United States
(12) Patent Application Publication
(10) Pub. No.: US 2012/0251443 A1
(43) Pub. Date: Oct. 4, 2012

(54) IMPLANTABLE CONTRAST AGENTS AND METHODS

(75) Inventors: C. Keith Ozaki, Brookline, MA (US); Jeffrey M. Karp, Brookline, MA (US); Frank Rybicki, Sudbury, MA (US); Dimitrios Mitsouras, Brookline, MA (US); Praveen Kumar Vemula, Cambridge, MA (US)

(73) Assignee: THE BRIGHAM AND WOMEN'S HOSPITAL, INC., Boston, MA (US)

(21) Appl. No.: 13/499,370

(22) PCT Filed: Oct. 1, 2010
(86) PCT No.: PCT/US10/51162

§ 371 Date: Jun. 8, 2012

(30) Foreign Application Priority Data
Oct. 2, 2009 (US) 61248404

(51) Int. Cl. A61K 49/00; A61K 51/02; G01N 21/64; A61K 49/08; A61K 51/04; G01N 24/08; A61K 49/04; A61K 49/06

(52) U.S. Cl. 424/1.65; 424/9.4; 424/1.11; 424/9.3; 424/9.6; 424/9.1; 424/9.32; 435/29

(57) ABSTRACT

The present disclosure provides compositions and methods based on implantable contrast agents that are useful for imaging tissue and organs.

### CROSS REFERENCE TO RELATED APPLICATION

This application claims priority to U.S. Provisional Patent Application No. 61/248,404, filed on Oct. 2, 2009, which is incorporated herein by reference in its entirety.

### STATEMENT REGARDING FEDERALLY SPONSORED RESEARCH

This invention was made with Government support under National Institutes of Health Grants No. HL095722 and HL099299. The Government has certain rights in this invention.

### TECHNICAL FIELD

The present disclosure relates to the design, synthesis, and use of implantable contrast agents and imaging of tissue and organs with such contrast agents.

### BACKGROUND

Frequently, clinicians need to image soft tissue structures such as blood vessels that have qualitative characteristics so similar to surrounding tissues that accurate resolution of the anatomy at the interface of the organs is currently impossible. In blood vessels, for example, current commercial contrast agents focus on lumen enhancement or parenchymal perfusion in the solid organs, and they often fail to delineate soft tissue interfaces.

### SUMMARY

Accordingly, the present disclosure provides, inter alia, compositions and methods of making and using implantable contrast agents that can be used to image cells, tissue, and/or organs (e.g., vein grafts), and compositions thereof. The implantable contrast agent can be covalently or non-covalently immobilized during surgery on the cells, tissue, and/or organs (e.g., vein grafts) of interest thereby enhancing and facilitating post-surgical clinical imaging. The implantable contrast agents include a group that is capable of attaching to the cell, tissue, and/or organ (e.g. vein grafts) of interest thereby enhancing tissue differentiation during clinical imaging. The implantable contrast agents can be detected by various imaging modalities including, e.g. single emission computed tomography (SPECT) scan, positron emission tomography (PET) scan, X-ray, computed tomography (CT), ultrasound (US), or magnetic resonance imaging (MRI) scan or other imaging modalities.

Accordingly, in a first aspect the present disclosure provides compositions for the ex vivo imaging of cells, tissue, and/or organs (e.g. vein grafts). The compositions include an isolated cell, tissue, vein graft, and/or organ and an implantable contrast agent that is covalently or non-covalently connected to the cell, tissue, and/or organ (e.g. vein or vein grafts).

In one aspect, the composition includes an isolated tissue or organ and a contrast agent that is immobilized thereon.

In another aspect, the method includes, contacting a tissue or organ of interest with a contrast agent capable of binding to the tissue or organ of interest, and allowing sufficient time for the contrast agent to be immobilized on the tissue or organ of interest; and obtaining an image of the tissue or organ of interest and one or more tissues or organs surrounding the tissue or organ of interest.

In a further aspect, the method of labeling a tissue or organ ex vivo or in vivo includes contacting the tissue or organ with a contrast agent capable of binding to the tissue or organ of interest, under conditions sufficient for the contrast agent to be immobilized on the tissue or organ.

In some embodiments, the organ of interest is part of the circulatory system e.g., a vein or a vein graft.

In some embodiments, the contrast agent is an X-ray contrast agent, computed tomography (CT) contrast agent, single photon emission computed tomography (SPECT) contrast agent, positron emission tomography (PET) contrast agent, infrared contrast agent, magnetic resonance imaging (MRI) contrast agent, or fluorescent dye.

In some embodiments, the contrast agent includes a metal chelator that chelates a detectable metal atom.

In some embodiments, the contrast agent is paramagnetic or radioactive.

In some embodiments, the contrast agent is a paramagnetic particle or ultra small paramagnetic particle, e.g., a superparamagnetic cross-linked iron-oxide (CLIO) particle and an iron oxide nanoparticle (FeNP).

In some embodiments, the contrast agent is immobilized on the tissue or organ via a covalent or non-covalent bond, e.g., via an amide bond, a thioamide bond, a thioester bond, and an ester bond.

In some embodiments, the method is carried out during, or as part of, a surgical procedure, e.g., surgically removing or implanting the tissue or organ, e.g., wherein the organ of interest is a transplant or a graft.

In some embodiments, the tissue or organ includes a functional group that is selected from the group consisting of an amino group, a thiol group, or a hydroxyl group.

In some embodiments, the contrast agent includes a functional group that is suitable for reacting with a functional group present on the tissue or organ, e.g., an activated ester, e.g., an N-hydroxysuccinamide (NHS) ester.

In some embodiments, the methods described herein include obtaining a plurality of images, e.g., obtained over time.

In some embodiments, the image includes detecting the contrast agent.

In some embodiments, the methods includes detecting the contrast agent using an imaging modality selected from X-ray, computed tomography (CT), positron emission tomography (PET), magnetic resonance imaging (MRI), ultrasound, and single photon emission computed tomography (SPECT), thereby obtaining an image of the tissue or organ of interest and one or more tissues or organs surrounding the tissue or organ of interest.

In some embodiments, the methods and compositions described herein include a kit which includes an implantable contrast agent with a carboxylic acid group, and reagents for activating the carboxylic acid group to an activated ester group.

In some embodiments, the organ is a vein or a vein graft.

In some embodiments, the contrast agent is immobilized on the tissue or organ by forming a covalent or non-covalent bond with the tissue or organ, e.g., by activating a carboxylic acid group on the contrast agent to form an activated ester; and reacting the contrast agent having the activated ester with an amine group, thiol group, or a hydroxyl group present on the tissue or organ (e.g., part of a protein or other moiety naturally present on the tissue or organ or added thereto) to form an amide bond, thioester bond, or an ester bond, respectively.

In some embodiments, the detectable metal atom is selected from gadolinium, dysprosium, indium-111, Technetium-99m, copper-64, and gallium-6; In some embodiments, the contrast agent is Gadopentetic acid, Gadobenic acid, Gadoteridol, Gadoxetic acid, Gadobutrol, Gadocoletic acid, or a Gadoteric acid.

In some embodiments, the methods described herein can be used for monitoring vein graft morphology in a subject.

By virtue of their design, the implantable contrast agents described herein possess certain advantages and benefits. First, the immobilized contrast agents provide for more efficient imaging and larger craniocaudal coverage. Second, because these contrast agents are permanently attached to the cell, tissue or organ (e.g. vein graft) of interest they allow for non-invasive and enhanced imaging, e.g. better definition of the boundaries of the cell, tissue, and/or organ (e.g. vein grafts) of interest. For example, by enhancing the imaging sensitivity to clearly delineate an entire vessel wall, the relationship between vein wall remodeling and early events (first four weeks post implantation) may eventually be investigated longitudinally and more efficiently. Third, since negative wall remodeling (permanent constriction of the vein graft) stands as an important etiology of graft failure, more sensitive delineation of early negative remodeling may also enable timely clinical interventions to halt the process. Additionally, the implantable contrast agents are nontoxic, biocompatible, and robust and remain bonded to the cells, tissue or organs of interest over an extended period of time thus facilitating regular post-surgical evaluation.

Unless otherwise defined, all technical and scientific terms used herein have the same meaning as commonly understood by one of ordinary skill in the art to which this invention belongs. Exemplary methods and materials are described herein; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages will be apparent from the following detailed description and figures, and from the claims.

### BRIEF DESCRIPTION OF THE DRAWINGS

FIG. 1 is a schematic of the immobilization of Fe-NPs on human vein.

FIGS. 2A and 2B are scanning electron microscopy (SEM) images of the outer surface of a human vein with immobilized Fe-NPs.

FIGS. 2C and 2D are scanning electron microscopy (SEM) images of the outer surface of a human vein without Fe-NPs.

FIGS. 3A and 3B are SEM images of unlabeled and Fe-NPs labeled surface of human veins, respectively.

FIGS. 3C and 3D are energy dispersive X-ray (EDAX) spectrometry images of unlabeled and Fe-NPs labeled surface of human veins, respectively.

FIGS. 4A and 4B are Perl's Prussian blue images of Fe-NPs labeled and unlabeled human veins.

FIGS. 5A-O are T1-weighted, T2*-weighted, and PD-weighted MR images obtained at 3 Tesla of three segments of the same saphenous vein labeled with activated Fe-NPs at varying concentrations and the unlabeled control.

FIGS. 6A-C are magnetic resonance (MR) images of definitions of three regions-of-interest (ROIs) and lengths used for quantitative analyses used to determine the average thickness of the vessel wall, the average thickness of the Fe-NP label, and the contrast-to-noise ratio (CNR) between vessel wall and surrounding label or saline.

FIG. 7 is a graph of average wall thickness of labeled and unlabeled (control) vein segments.

FIG. 8 is a line graph of the correlation between label thickness and Fe-NP load observed in a dose-response experiment.

FIG. 9 is a schematic of the activation of carboxylic acid groups of a Gadopentetate Dimeglumine (Gd-DTPA) complex using N-hydroxysuccinamide chemistry and labeling of a vein.

FIGS. 10A and 10B are 3-dimensional (3D) Spoiled GRASS(SPGR) T1W images of saphenous vein labeled with activated Gd-DTPA and without labeling, respectively.

FIGS. 10C and 10D are ESEM and EDAX images of a labeled vein confirming the presence of Gadolinium (Gd).

FIGS. 11A-K are 3D T1W SPGR T1W images and T_{1,2} relaxation times of veins incubated in varying concentrations of activated and non-activated Gd-DTPA.

FIGS. 12A and B are line graphs of traverse and longitudinal relaxation rates of two tissue specimens.

FIG. 13A is a MR image of T₂ maps of a vein segment labeled with the implantable Gd-DTPA contrast agent and an untreated control.

FIGS. 13B and C are line graphs depicting mean R₂ and R₁ rates for each segment across time points.

### DETAILED DESCRIPTION

The present disclosure features new compositions and methods for labeling cells, tissues, vein grafts, and/or organs, e.g., for in vivo imaging methods that enable enhanced visualization of the cells, tissues, vein grafts, and/or organs within a patient. In some embodiments, the method is performed by covalently or non-covalently binding an implantable contrast agent to the cells, tissues, vein graft, and/or organs (e.g., at the time of surgery) thereby allowing enhanced imaging of the cells, tissues and/or organs (e.g. a vein graft) (Scheme 1). The implantable contrast agent can include a functional group, e.g. a group that can attach to a handle (“Y” in Scheme 1) (e.g., an amine group, thiol group, or hydroxyl group) found on the cells, tissues, vein graft, and/or organs, e.g., on an extracellular matrix of any thereof. After modification of the functional group on the implantable contrast agent has been achieved this “activated” contrast agent can be contacted with the cells, tissues, vein graft, and/or organs to form a permanent chemical bond (e.g. amide, thioester, ester, respectively) (Scheme 1). Thereafter, the implantable contrast agent can be detected on cells, tissue, and/or organs of interest in vivo by various techniques and systems including MRI, SPECT, PET, CT, IR, US, X-ray, and other imaging modalities.

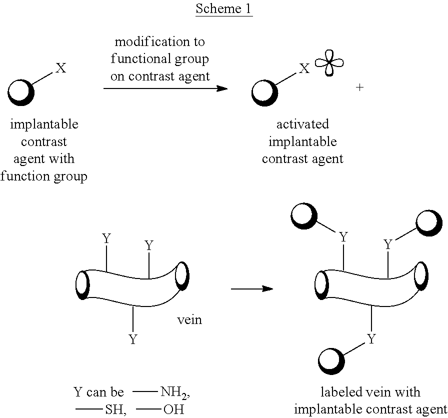

### Cells, Tissues and Organs

In some embodiments, the cell, tissue or organ can be part of the skeletal, muscular, circulatory, nervous, respiratory, digestive, excretory, endocrine, reproductive, or lymphatic or immune system. For example, the methods described herein can be useful for labeling components of a patient's circulatory system, e.g., a patient's vasculature. The term “vasculature” as used herein refers to the vascular system (or any part thereof) of a body, human or non-human, and includes blood vessels, e.g., arteries, arterioles, veins, venules, and capillaries. As another example, the methods described herein are useful for labeling a vein graft prior to implantation in a patient.

The term “cell(s)” or “animal cell(s)” as used herein refers to any type of animal cells. By “isolated cell” is meant that the cell is removed from the tissue or organ in which it (or its predecessor) naturally occurs. A cell can be just partially purified from its natural milieu and be deemed “isolated.” The cells of an intact organ such as a kidney or heart or a partial organ such as a piece of a blood vessel are not considered to be “isolated cells” while still part of the organ.

The term “organ(s)” is used throughout the specification as a general term to describe any anatomical part or member having a specific function in the animal. Included in this definition are blood vessels and vein grafts. Further included within the meaning of this term are substantial portions of organs, e.g., cohesive tissues obtained from an organ. Such organs include but are not limited to kidney, liver, heart, intestine, e.g., large or small intestine, pancreas, and lungs. Further included in this definition are bones and skin.

The term “patient” is used throughout the specification to describe an animal, human or non-human, to whom treatment and/or imaging according to the methods of the present disclosure is provided. Veterinary and non-veterinary applications are contemplated. The term includes but is not limited to mammals, e.g., humans, other primates, pigs, rodents such as mice and rats, rabbits, guinea pigs, hamsters, cows, horses, cats, dogs, sheep and goats.

The term “contacting” or “contacted” is used throughout the specification to describe the act of applying the implantable contrast agent to the organ or tissue or interest. For example, contacting the tissue or organ can mean the act of “painting” or spraying the implantable contrast agent onto the tissue or organ of interest during a surgical procedure. The “painting” or spraying can be performed ex vivo or in vivo. In the case where the contacting occurs ex vivo, the contacting can occur just prior to or during the surgical procedure.

### Implantable Contrast Agents

The implantable contrast agent can be any contrast agent amenable to the procedures described herein, e.g., a positive or negative contrast agent capable of attaching to a cell, tissue, or organ (e.g. vein graft) and capable of being imaged by clinical imaging modalities. For example, the implantable contrast agent can be a positive contrast agent. In some embodiments, the contrast agent can include a magnetic resonance imaging moiety that includes a chelator moiety and a chelated paramagnetic or superparamagnetic metal atom or ion.

Exemplary paramagnetic metals include Dy and Gd. Exemplary radioactive metals include, but are not limited to ¹¹¹In, ^{99m}Tc, ⁶⁴Cu, and ⁶⁸Ga. Nonmetals like radioactive halides or ¹¹C can also be used. Table 1 below demonstrates various metals and nonmetals that can be used as part of an implantable contrast agent and their method of detection. Examples of radiolabeled atoms include, but are not limited to ¹⁸F, ¹⁴C, and ³H.

TABLE 1
Exemplary Atoms that can be used
with Implantable Contrast Agents

| Atom | Imaging Modality |
|---|---|
| Dy | MRI |
| Gd | MRI |
| Iron (superparamagnetic | MRI |
| iron oxide) | |
| ¹¹¹In | SPECT |
| ⁶⁴Cu | PET |
| ⁶⁸Ga | PET |
| ¹⁸F | PET |
| ¹²³I | SPECT |
| ¹²⁴I | PET |
| ⁸⁹Zr | PET |
| ⁸⁶Yt | PET |
| ¹¹C | PET |
| ^{99m}Tc | SPECT |

Various chelating moieties are known, and can be incorporated into an implantable contrast agent. In addition, novel chelating moieties can be discovered in the future and can be incorporated into an implantable contrast agent. In certain embodiments, the chelating moiety does not form a covalent bond with the paramagnetic or superparamagnetic metal or metal oxide. In some embodiments, the chelating moiety forms a thermodynamically and kinetically stable, non-covalent coordination complex or ionic complex with (iron) Fe³⁺, (gadolinium) Gd³⁺, (dysprosium) Dy³⁺, (europium) Eu³⁺, (manganese) Mn²⁺, or other useful metals (e.g., praseodymium (Pr), neodymium (Nd), samarium (Sm), terbium (Tb), holmium (Ho), erbium (Er), thulium (Tm), ytterbium (Yb), or lutetium (Lu), or an ion or metal oxide thereof). In certain embodiments, the metal atom or ion is (gadolinium) Gd³⁺.

In some embodiments, the chelator can be 1,4,7,10-tetraazacyclodo-decane-N,N′,N″,N′″-tetraacetic acid; 1,4,7,10-tetraaza-cyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N′,N″,N′″-tetraacetic acid; diethylenetriamine-pentaacetic acid (DTPA); ethylenedicysteine; bis(aminoethanethiol)carboxylic acid; triethylenetetraamine-hexaacetic acid; ethylenediamine-tetraacetic acid (EDTA); 1,2-diaminocyclohexane-N,N,N′,N′-tetraacetic acid; N-(hydroxy-ethyl)ethylenediaminetriacetic acid; nitrilotriacetic acid; and ethylene-bis(oxyethylene-nitrilo)tetraacetic acid.

In some embodiments, the implantable contrast agent can include a small molecule with a molecular weight of less than about 800 Daltons. For example, the implantable contrast agent can be Gd-DTPA (cation MW=547 daltons) and because of its low molecular weight can quickly enter the interstitial space and enhance the entirety of the vessel wall tissue. When a vein is incubated with the activated Gd chelate, the complex can readily permeate the fibrous adventitial layer through the vasa vasorum, subsequently reacting with handle found deeper within the tissue constituents to effectively become permanently “immobilized” there.

In some embodiments, the contrast agent can be a negative contrast agent. In embodiments, the contrast agent can include a magnetic resonance imaging moiety that includes a magnetic nanoparticle, (e.g., magnetic metal oxide, such as superparamagnetic iron oxide). In certain embodiments, the magnetic nanoparticle can be a small paramagnetic iron oxide (SPIO) or an ultra-small paramagnetic iron oxide (USPIO). In certain embodiments, the magnetic nanoparticle can be magnetic iron oxide nanoparticles (Fe-NPs), or a coated, cross-linked iron oxide (e.g., an iron oxide nanoparticle coated with amines, cross-linked dextran, e.g., (CLIO)). The magnetic metal oxide can also comprise cobalt, magnesium, zinc, or mixtures of these metals with iron. The term “magnetic” as used herein means materials of high positive magnetic susceptibility such as superparamagnetic compounds and magnetite, gamma ferric oxide, or metallic iron. In certain embodiments, the magnetic nanoparticle can have a relatively high relaxivity, e.g., strong effect on water relaxation.

In some embodiments, the implantable contrast agent can be a biologically based contrast agent. Examples can include microparticles of iron oxide (MPIOs) conjugated to a single-chain antibody specific for a ligand-induced binding site (LIBS) (LIBS-MPIO), and gadolinium-based collagen-targeting contrast agent (e.g., EP-3533). Other examples can be found in Waters, E. A. et al. Basic research in cardiology 103: 114-121 (2008); Jaffer F. A. et al. Circulation 116: 1052-1061 (2007); von Zur Muhlen, C. et al. The Journal of Clinical Investigation 118: 1198-1207 (2008); Caravan, P. Angewandte Chemie International Ed. 46: 8171-8173 (2007); Helm, P. A. et al. Radiology 247: 788-796 (2008); Yamamoto, T. Bioorganic & Medicinal Chemistry Letters 14: 2787-2790 (2004); Yamamoto, T. et al. Anal. Sci. 20: 5-7 (2004); and Jaffer, F. A. et al. Circulation 118: 1802-1809 (2008).

In some embodiments, the implantable contrast agent can include an NIR contrast agent that fluoresces in the near infrared region of the spectrum. Exemplary near-infrared fluorophores can include dyes and other fluorophores with emission wavelengths (e.g., peak emission wavelengths) between about 680 and 1000 nm, e.g., between about 680 and 800 nm, between about 800 and 900 nm, between about 900 and 1000 nm, between about 680 and 750 nm, between about 750 and 800 nm, between about 800 and 850 nm, between about 850 and 900 nm, between about 900 and 950 nm, or between about 950 and 1000 nm. Fluorophores with emission wavelengths (e.g., peak emission wavelengths) greater than 1000 nm can also be used in the methods described herein. Exemplary fluorophores include indocyanine green (ICG), IRDye78, IRDye80, IRDye38, IRDye40, IRDye41, IRDye700, IRDye800, Cy5.5, Cy7, Cy7.5, IR-786, DRAQ5NO (an N-oxide modified anthraquinone), quantum dots, and analogs thereof, e.g., hydrophilic analogs, e.g., sulfonated analogs thereof. Commercially obtainable fluorophores include dyes such as IRDye® 78, 680, and 800CW infrared dyes (LI-COR Biotechnology, Lincoln, Nebr.), Alexa® Fluors 680, 700, and 750 (Invitrogen, Carlsbad, Calif.), and CyDye™ Fluor Cy5.5, Cy7, and Cy7.5 (GE Healthcare, Chalfont St. Giles, United Kingdom). Quantum dots with near-infrared emission can also be obtained commercially, such as Qdot® 705 and 800 nanocrystals (Invitrogen, Carlsbad, Calif.).

In some embodiments, the implantable contrast agent is a CT contrast agent or an implantable contrast agent that works by X-ray attenuation. These contrast agents can contain, e.g., iodine, barium, barium sulfate, and/or gastrografin.

In some embodiments, the implantable contrast agent can include contrast agents useful for ultrasonic (US) imaging (e.g., microbubble contrast agents). The implantable contrast agent can alter the echogenic properties of the labeled tissue or organ (i.e., more echo opaque). For example, the implantable contrast agent can enhance the echo imaging of the near wall of a vein, or for example, in the setting of a blood vessel and Intravascular Ultrasound (IVUS), can enhance the definition of the outer wall if previously labeled with an implantable contrast agent on the adventitia. Examples of microbubbles include Optison™ and Levovist® two FDA-approved microbubbles currently being used in echocardiography. Microbubbles are also described in Calliada, F. et al. “Ultrasound contrast agents: basic principles” European Journal of Radiology 27 Suppl. 2: S157-60 (1998) and in Lindner, J. R. et al. “Molecular imaging of myocardial and vascular disorders with ultrasound” JACC Cardiovasc. Imaging 3:204-11 (2010). Functional groups can be conjugated to microbubbles, e.g., by methods described in Willmann, J. K., et al. “Molecular imaging in drug development” Nat. Rev. Drug Discov. 7: 591-607 (2008) and in Willmann, J. K. et al. “Targeted Contrast-Enhanced Ultrasound Imaging of Tumor Angiogenesis with Contrast Microbubbles Conjugated to Integrin-Binding Knottin Peptides” Journal of Nuclear Medicine 51: 433-440 (2010).

### Particle

The implantable contrast agent can include a particle or alternatively a polymer that forms the core of the conjugates. These particles can be nanoparticles or microparticles. The term “nanoparticle” refers to a particle that has a characteristic dimension of less than about 1 micrometer, where the characteristic dimension is the largest cross-sectional dimension of a particle. For example, the particle may have a characteristic dimension of less than about 500 nm, less than about 250 nm, less than about 150 nm, less than about 100 nm, less than about 50 nm, less than about 30 nm, less than about 10 nm, or less than about 3 nm in some cases. Microparticles with a size of between 1.0 μm and 100 μm can also be employed in the new conjugates. In some embodiments, the polymer can have a molecular weight from 2 to 2,000 Kilodaltons.

The particles or polymers useful in the methods and compositions described herein can be made of materials that have one or more of the following properties: (i) are biocompatible, e.g., do not cause a significant adverse reaction in a living animal when used in pharmaceutically relevant amounts; (ii) feature functional groups to which the implantable contrast agent can be covalently or non-covalently attached to the cell, tissue, or organ (e.g. vein graft); (iii) exhibit low non-specific binding of interactive moieties to the particles; and (iv) are stable in solution, e.g., the particles or polymers do not substantially degrade or precipitate. The particles or polymers can be monodisperse (e.g., a single crystal of a material, e.g., a metal, per particle, or have a discrete molecular weight) or polydisperse (e.g., particles with range of different diameters, or polymers with a range of molecular weights).

A number of particles are known in the art, e.g., organic or inorganic nanoparticles. Liposomes, dendrimers, carbon nanomaterials, and polymeric micelles are examples of organic nanoparticles. Inorganic particles include metallic particles, e.g., Au, Ni, Pt and TiO₂ particles. For example, magnetic nanoparticles can also be used, e.g., spherical nanocrystals of 10-20 nm with a Fe²⁺ and/or Fe³⁺ core surrounded by dextran or PEG molecules. In some embodiments, colloidal gold nanoparticles are used, e.g., as described in Qian et al., Nat. Biotechnol. 26: 83-90 (2008); U.S. Pat. Nos. 7,060,121; 7,232,474; and U.S. P.G. Pub. No. 2008/0166706. Suitable multifunctional nanoparticles, and methods for constructing and using multifunctional nanoparticles, are discussed in e.g., Sanvicens and Marco, Trends Biotech., 26:425-433 (2008). A number of different polymers are known in the art. These can include, but are not limited to dextrans, aminodextrans, carboxymethyldextrans, carboxymethyl starches, polyvinyl alcohols, polyethylene glycols, poly-lysines, or poly-glutamic acids.

The particles or polymers can be attached to the cell, tissue or organ of interest via a functional group, e.g. carboxylic acid or ester. In some embodiments, the particles are associated with a polymer that includes the functional groups, and also serve to keep the metal oxides dispersed from each other. The polymer can be a synthetic polymer, such as, but not limited to, polyethylene glycol or silane, natural polymers, or derivatives of either synthetic or natural polymers or a combination of these. In some embodiments, the polymers are hydrophilic. In some embodiments, the polymer “coating” is not a continuous film around the magnetic metal oxide, but is a “mesh” or “cloud” of extended polymer chains attached to and surrounding the metal oxide. The polymer can comprise polysaccharides and derivatives, including dextran, pullanan, aminodextran, carboxmethyl dextran, and/or reduced carboxymethyl dextran. The metal oxide can be a collection of one or more crystals that contact each other, or that are individually entrapped or surrounded by the polymer.

In some embodiments, the particles are associated with non-polymeric functional group compositions. Methods are known to synthesize stabilized, functionalized particles without associated polymers, which are also within the scope of this disclosure. Such methods for use to make nanoparticles are described, for example, in Halbreich et al., Biochimie, 80:379-390, 1998.

In some embodiments, the nanoparticles have an overall size of less than about 1-100 nm, e.g., about 25-75 nm, e.g., about 40-60 nm, or about 50-60 nm in diameter. The polymer component in some embodiments can be in the form of a coating, e.g., about 5 to 20 nm thick or more. The overall size of the nanoparticles is about 15 to 200 nm, e.g., about 20 to 100 nm, about 40 to 60 nm; or about 60 nm.

In embodiments, the implantable contrast agent can be immobilized on the outer surface of the tissue or organ (e.g., vessel wall) versus through some thickness (e.g., throughout vessel wall) based on size of nanoparticles (e.g., size range between about 5 nm and about 10,000 nm.

In embodiments, the particles or polymers can be small enough (e.g. less than 500 nm) to penetrate the tissue or organ to react with the handle that are located deeper within the tissue or organ.

### Synthesis of Particles

There are varieties of ways that the particles can be prepared, e.g., known in the art and/or described herein, but in all methods, the result must be a particle with functional groups that can be used to attach the implantable contrast agent to the cell, tissue, or organ of interest.

There are several methods for placing a functional group, such as a carboxy, on the particle. Methods for synthesizing functionalized, coated particles are discussed in further detail below.

Carboxy functionalized nanoparticles can be made, for example, according to the method of Gorman (see WO 00/61191). Carboxy-functionalized nanoparticles can also be made from polysaccharide coated nanoparticles by reaction with bromo or chloroacetic acid in strong base to attach carboxyl groups. In addition, carboxy-functionalized particles can be made from amino-functionalized nanoparticles by converting amino to carboxy groups by the use of reagents such as succinic anhydride or maleic anhydride.

Carboxy-functionalized nanoparticles can be converted to amino-functionalized magnetic particles by the use of water-soluble carbodiimides and diamines such as ethylene diamine or hexane diamine.

Nanoparticle size can be controlled by adjusting reaction conditions, for example, by varying temperature as described in U.S. Pat. No. 5,262,176. Uniform particle size materials can also be made by fractionating the particles using centrifugation, ultrafiltration, or gel filtration, as described, for example in U.S. Pat. No. 5,492,814.

Nanoparticles can also be synthesized according to the method of Molday (Molday, R. S, and D. MacKenzie, “Immunospecific ferromagnetic iron-dextran reagents for the labeling and magnetic separation of cells,” J. Immunol. Methods, 1982, 52:353-67, and treated with periodate to form aldehyde groups. The aldehyde-containing nanoparticles can then be reacted with a diamine (e.g., ethylene diamine or hexanediamine), which will form a Schiff base, followed by reduction with sodium borohydride or sodium cyanoborohydride.

Dextran-coated nanoparticles can be made and cross-linked with epichlorohydrin. The addition of ammonia will react with epoxy groups to generate amine groups, see Hogemann, D., et al., “Improvement of MRI probes to allow efficient detection of gene expression” Bioconjug. Chem. 11: 941-6 (200), and Josephson et al., “High-efficiency intracellular magnetic labeling with novel superparamagnetic-Tat peptide conjugates,” Bioconjug. Chem. 10: 186-91 (1999). This material is known as cross-linked iron oxide or “CLIO” and when functionalized with amine is referred to as amine-CLIO or NH₂—CLIO.

In addition, other methods are known to covalently attach the implantable contrast agent to the tissue or organ of interest. For example, the implantable contrast agent can be further functionalized with a linker group wherein the linker group has a first end and a second end.

The term “linker” as used herein refers to a group of atoms, e.g. 5-100 atoms, and may be comprised of the atoms or groups of atoms, e.g. carbon, amino, alkylamino, oxygen, sulfur, sulfoxide, sulfonyl, amide, and carbonyl. The linker is connected at a first end to the cell, tissue or organ of interest through an amide bond, an ester bond, or through a thioester bond, e.g. through a carboxylic acid/ester or activated carboxylic acid on the linker reacts with the handle (e.g., amine, hydroxyl, or thiol) of the cell, tissue or organ of interest. In addition, the linker is connected at a second end to the implantable contrast agent through a covalent bond. Examples of covalent bonds include, but are not limited to an amide bond or a triazole ring. Alternatively, a first linker can be extended with a second linker, to produce a longer and more complex linker, prior to attachment of the reporter group. In embodiments, the linker can be used to further enhance or aid in the imaging of the cell, tissue or organ of interest. In embodiments where the methods include a linker, the linker is first attached to the implantable contrast agent and then the implantable contrast agent with the attached linker will then attach to the cell, tissue, or organ.

For example, the first end of the linker group can have an activated ester that can react with the amino group, hydroxyl group, or thiol group on the tissue or organ forming a covalent bond (amide, ester, thioester, respectively). The second end of the linker group can also have a functional group appropriate for attachment to the contrast agent through “click” chemistry (see, e.g., the Sigma Aldrich catalog and U.S. Pat. No. 7,375,234, which are both incorporated herein by reference in their entireties). Of the reactions including “click” chemistry, one example is the Huisgen 1,3-dipolar cycloaddition of alkynes to azides to form 1,4-disubstituted-1,2,3-triazoles. The copper (I)-catalyzed reaction is mild and very efficient, requiring no protecting groups, and requiring no purification in many cases. The azide and alkyne functional groups are generally inert to biological molecules and aqueous environments.

In some embodiments, the implantable contrast agent (or each of the implantable contrast agents) can be attached to the tissue or organ (e.g., organ) of interest by forming a covalent bond between a functional group that is attached to the organ of interest and a functional group that is attached to the implantable contrast agent.

In certain embodiments, the functional group that is attached to the organ of interest is an amino group, and the functional group that is attached to the implantable contrast agent is an activated carboxylic acid.

As used herein, the term “activated carboxylic acid” refers to a derivative of a carboxyl group that is more susceptible to nucleophilic attack than a free carboxyl group; e.g., acid anhydrides, thioesters, and esters (e.g., an NHS ester).

Synthetic chemistry transformations useful for introducing and reacting such functional groups are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Additionally, the linker can be a cleavable linker placed between the implantable contrast agent and the tissue or organ. Such a linker can be resistant to cleavage by enzymes present in the plasma, tissue or organ. After the implantable contrast agent is no longer needed, a cleaving agent (e.g., a protease) can be administered to the patient “on demand” to cleave the specific linker, resulting in the release of the implantable contrast agent, e.g., in a form that can be cleared rapidly by the kidneys. Examples can include TNKase® and linkers containing disulfide bonds (S—S bond) that can be rapidly cleaved in vivo. In some embodiments, the cleavable linker can be sensitive to light and can thus be cleaved using light (e.g. UV light).

### Methods of Detecting Implantable Contrast Agents

The implantable contrast agents of the present disclosure can be imaged in vivo by single emission computed tomography (SPECT) scan, positron emission tomography (PET) scan, X-ray computed tomography (CT), ultrasound (US) or magnetic resonance imaging (MRI) scan or other imaging modalities either alone or in combination with other traditional imaging modalities such as NIR imaging. The implantable contrast agents can be imaged by these imaging modalities to enhance post-surgical tissue differentiation. The implantable contrast agent attached to the tissue or organ of interest can be imaged by these modalities such that the portion of the image associated with the tissue or organ of interest visually contrasts with the portion of the image associated with the tissue or organ surrounding the tissue or organ of interest, thereby visually differentiating the tissue or organ of interest from the one or more tissues or organs surrounding the tissue or organ of interest. Additionally, these implantable contrast agents can be detected by traditional fluorescence imaging techniques allowing for the facile tracking of the implantable contrast agents by fluorescence microscopy or flow cytometry using methods known in the art, e.g., as described in US 2005/0249668.

In addition, the compositions and methods of the present disclosure can be used in combination with other imaging compositions and methods. For example, the implantable contrast agents of the present disclosure can be imaged by MR imaging methods either alone or in combination with other traditional imaging modalities, such as, X-ray, computed tomography (CT), NIR imaging, ultrasound, positron emission tomography (PET), and single photon computerized tomography (SPECT). For instance, the methods described herein can be used in combination with CT to obtain both anatomical and molecular information simultaneously, for example, by co-registration of with an image generated by another imaging modality.

### Uses of Contrast Agents

### In Vivo Imaging

The implantable contrast agents described herein can be used in in vivo imaging methods to visualize organs, cells, and other structures. In some embodiments, the contrast agents are used to identify and evaluate vein graft morphology (graft failure), injury, and necrosis. Such methods can include administering to a subject one or more implantable contrast agents described herein; allowing the implantable contrast agent to form a covalent or non-covalent bond with the tissue or organ of interest within the subject; and imaging the subject by MRI, PET, SPECT, CT, X-ray, US, NIR, and/or other imaging modality to visually differentiate the tissue or organ of interest in the subject from one or more tissues or organs surrounding the tissue or organ of interest in the subject. Furthermore, it is understood that the methods (or portions thereof) can be repeated at intervals to evaluate the subject over time, e.g. determine vein graft acceptance or rejection.

Information provided by such in vivo imaging, for example, the presence, absence, or level of emitted signal, can be used to detect and/or monitor tissue damage, inflammation, and/or disease in the subject. Examples of causes of tissue damage include, without limitation, Alzheimer's disease, atherosclerosis, cancer, stroke, inflammatory bowel disease, diabetes, and organ transplant rejection. In addition, in vivo imaging can be used to assess the effect of a compound or therapy by using the implantable contrast agents, wherein the subject is imaged prior to and after treatment with the compound or therapy, and the corresponding signal/images are compared. For example, a subject can be imaged prior to removal of a tissue or organ of interest and after re-implantation of the tissue or organ of interest that has been treated with an implantable contrast agent.

In embodiments, the methods described herein can be used, e.g., for solid organ labeling, labeling of aneurysms, labeling heart ventricles, and labeling tumor beds after resection.

In embodiments, the methods described herein can be used for earlier detection of peripheral artery disease or coronary occlusive disease through the ability to temporally interrogate the in vivo vein graft wall anatomy.

In embodiments, the methods described herein can be used for efficient longitudinal solid organ topographical imaging in primary atherosclerosis, restenosis after angioplasty, dialysis access failure, and in fields such as oncology (e.g. to aid in survey for cancer recurrence after surgical resection). Other embodiments include use of the implantable contrast agent to coat a tissue or organ for imaging (e.g., to monitor tumor regrowth after resection, bowel motility after surgery).

In embodiments, the methods described herein can be useful for imaging the ureters during abdominal surgeries such as caesarian sections and urological surgeries, where it is crucial that the surgeon be able to identify the ureters, e.g., to avoid damaging them, or to repair them after iatrogenic or external damage. These methods can include injecting the contrast agent via direct cannulation, either anterograde or retrograde, into the ureters or bladder, such that it appears in the urine stream.

In embodiments, the methods described herein can be used to image the biliary tree by injecting the implantable contrast agent via direct cannulation into the right or left hepatic duct for anterograde or retrograde labeling of the biliary tree. The methods and compositions described herein can be used to help a physician or surgeon to identify and characterize areas of disease, such as the early events in blood vessel wall morphology. For example, implantable contrast agents can be used for vascular imaging techniques to study total vein graft wall adaptations and remodeling.

The methods and compositions disclosed herein can also be used to monitor and/or guide various therapeutic interventions, such as surgical procedures, and monitoring drug therapy, including cell based therapies. The methods can also be used in prognosis of a disease or disease condition.

With respect to each of the foregoing, examples of such disease or disease conditions that can be detected and/or monitored (before, during or after therapy) include inflammation (for example, inflammation caused by arthritis, for example, rheumatoid arthritis), cancer (for example, colorectal, ovarian, lung, breast, prostate, cervical, testicular, skin, brain, gastrointestinal, pancreatic, liver, kidney, bladder, stomach, leukemia, mouth, esophageal, bone), cardiovascular disease (for example, atherosclerosis and inflammatory conditions of blood vessels, ischemia, hypertension, stroke, myocardial infarction, thrombosis, disseminated intravascular coagulation), infectious disease (for example, bacterial, viral, fungal and parasitic infections, including Acquired Immunodeficiency Syndrome, Malaria, Chagas Disease, Schistosomiasis), immunologic disease (for example, an autoimmune disorder, lymphoma, multiple sclerosis, rheumatoid arthritis, diabetes mellitus, lupus erythematosis, myasthenia gravis, Graves disease), and surgery-related complications (such as organ rejection, alterations in wound healing, fibrosis or other complications related to surgical implants).

The methods described herein, therefore, can be used, for example, to determine the presence and localization of vascular disease including areas at risk for acute occlusion (e.g., vulnerable plaques) in coronary and peripheral arteries, regions of expanding aneurysms, and ischemic areas. The methods can also be used for drug delivery and to monitor drug delivery, especially when drugs or drug-like molecules are chemically attached to the implantable contrast agents.

### In Vitro Imaging

With respect to in vitro/ex vivo imaging methods, the implantable contrast agents described herein can be used in a variety of in vitro assays. An exemplary in vitro imaging method comprises: contacting a sample, for example, a biological sample, e.g. vein graft, with one or more implantable contrast agents described herein; allowing the implantable contrast agent(s) to interact with the biological sample; optionally, removing unbound implantable contrast agents; obtaining micrographs of the biological samples using environmental scanning electron microscope (ESEM); mapping by elemental mapping analysis the biological sample to identify the presence of the implantable contrast agent on the biological sample using an energy dispersive X-ray (EDAX) spectrometry; and detecting a signal emitted from the implantable contrast agent thereby determining whether the implantable contrast agent is bound to the biological sample.

### Compositions

The invention further comprises preparations, formulations, kits, and the like, comprising any of the compositions as described herein for use in various methods. In some embodiments, the kit can include instructions for use of the kits in the methods described herein. “Instructions” can define a component of promotion, and typically involve written instructions on or associated with packaging of compositions of the invention. Instructions also can include any oral or electronic instructions provided in any manner. The “kit” typically defines a package including any one or a combination compositions described herein and the instructions, but can also include the compositions described herein and instructions of any form that are provided in connection with the composition in a manner such that a clinical professional will clearly recognize that the instructions are to be associated with the specific composition.

The kits described herein can also contain one or more containers, which can contain compounds, e.g., as described herein. The kits also can contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit can be provided as any suitable form, for example, as liquid solutions or as dried powders. When the composition provided is a dry powder, the powder can be reconstituted by the addition of a suitable solvent, which can also be provided. In embodiments where liquid forms of the composition are sued, the liquid form can be concentrated or ready to use. The solvent can depend on the compound and the mode of use or administration. Suitable solvents for drug compositions are well known and are available in the literature.

The kit, in one set of embodiments, can comprise a carrier means being compartmentalized to receive in close confinement one or more container means such as vials, tubes, and the like, each of the container means comprising a specific composition. Additionally, the kit can include containers for other components, for example, buffers useful in the assay.

The implantable contrast agents described herein can be delivered to the tissue or organ of interest either in vivo or ex vivo dry or dissolved in a carrier or vehicle, e.g., pharmaceutically acceptable carriers and vehicles. Useful carriers and vehicles include, but are not limited to, buffer substances such as phosphate, glycine, sorbic acid, potassium sorbate, tris(hydroxymethyl)amino methane (“TRIS”), partial glyceride mixtures of fatty acids, water, salts or electrolytes, disodium hydrogen phosphate, potassium hydrogen phosphate, and sodium chloride.

The implantable contrast agents can be administered in the form of a sterile preparation. The possible vehicles or solvents that can be used to make injectable preparations include water, Ringer's solution, and isotonic sodium chloride solution, and 5% D-glucose solution (D5W). In addition, oils such as mono- or di-glycerides and fatty acids such as oleic acid and its derivatives can be used. The implantable contrast agents and compositions thereof can be administered by applying directly to the tissue or organ of interest. The implantable contrast agents can also be administered via catheters or through a needle to any tissue.

Application of the implantable contrast agent will depend on a number of factors including the sensitivity of the detection system used, e.g. the choice of implantable contrast agent and the imaging modality, as well as a number of subject-related variables, including animal species, age, body weight, mode of administration, sex, diet, time of administration, and rate of excretion.

Prior to use of the methods and compositions described herein, the subject can be treated with an agent or regimen to enhance the imaging process. For example, a subject can be put on a special diet prior to imaging to reduce any auto-fluorescence or interference from ingested food, such as a low pheophorbide diet to reduce interference from fluorescent pheophorbides that are derived from some foods, such as green vegetables. Alternatively, a cleansing regimen can be used prior to imaging, such as those cleansing regimens that are used prior to colonoscopies and include use of agents such as Visiciol™. The subject (patient or animal) can also be treated with pharmacological modifiers to improve image quality. For example, using low dose enzymatic inhibitors to decrease background signal relative to target signal (secondary to proportionally lowering enzymatic activity of already low-enzymatic activity normal tissues to a greater extent than enzymatically-active pathological tissues) can improve the target-to-background ratio during disease screening.

### Exemplary Methods

In certain methods of the present disclosure, implantable contrast agents can be immobilized on a cell, tissue, and/or organ. For example, a vein graft outer wall can be functionalized with a negative implantable contrast agent (e.g., iron oxide particles, Fe-NPs), and/or a positive implantable contrast agent (e.g., Gd-DTPA). Both techniques can involve activation of carboxylic acid using N-hydroxysuccinamide chemistry, followed by attaching to the handle (e.g., amine, hydroxyl, thiol) on the vein graft surface.

The invention is illustrated in part by the following examples, which are not to be taken as limiting the invention in any way.

### EXAMPLES

### Example 1

### Immobilized Iron Oxide Magnetic Nanoparticles (Fe-NPs) for Enhancement of Vessel Wall Magnetic Resonance Imaging

### Experimental Details of the Preparation and Characterization of Fe-NP Labeled Veins

Acquisition of Vein Specimens and Vein Conduit Labeling:

Fresh discarded human vein segments (n=3) from bypass surgeries and major amputations were collected by following approved institutional protocols and used for labeling experiments. The sample size used in this proof of principle experiment (n=3 total) was small, and was largely limited by the scarcity of adequately sized fresh discarded human vein specimens.

Fe-NPs that have carboxylic acid groups on the surface (˜30 nm) were purchased from Ocean Nanotech LLC USA. The surface carboxylic acid groups were activated through the N-hydroxysuccinamide ester reaction as follows: 50 μL of Fe-NPs were (5 mg/mL water) diluted with 150 μL of double distilled water (ddH₂O). The particle solution was centrifuged at 13,000 rpm for 6 minutes. The supernatant was removed and 200 μL of dimethyl formamide (DMF) was added, then the solution was vortexed and centrifuged for 6 minutes. The supernatant was again removed, and the pellet re-dispersed in 200 μL of DMF containing N,N′-diisopropylcarbodiimide (DIC, 1 mmol/L) and N-hydroxysuccinamide (1 mmol/L). The reaction continued at room temperature overnight.

The activated particles including the activated ester were then spun down and washed with DMF (200 μL). The particles were finally re-dispersed in phosphate buffer saline (PBS) and used immediately for coating the vein graft adventitia. With the luminal side protected via ligatures on either end, the test human vein sample (n=1) was placed in a PBS filled Petri-dish. The activated Fe-NPs (200 μL in PBS) were added and incubated for 30 minutes at room temperature. The procedure was repeated for the unlabeled control vein segment (n=1), but incubated with plain PBS without the addition of Fe-NPs. Finally, all vein segments were thoroughly rinsed thrice with PBS to remove nonspecifically bound nanoparticles. Different sets of instruments were employed for each study group to avoid cross contamination.

Dose-Responsive Samples:

To non-covalently fixate different concentrations of Fe-NPs on the vein, four concentrations of Fe-NPs (50, 25, 12.5 and 6 μg) were each mixed with a drop of Cyanoacrylate adhesive and placed on the adventitia of a segment of unlabeled vein (n=1) prior to imaging. This method resulted in non-covalent localization of Fe-NPs on the vein.

Evaluation of Contrast Localization—SEM and EDAX:

Scanning Electron Microscopy (SEM) with energy dispersive spectroscopy (EDAX) was used to map the presence of Fe-NPs on the surface of the vein following MR imaging. The Fe-NP-coated vein segments were mounted on an aluminum stub with carbon tape, and directly examined using Environmental SEM (FEI/Phillips XL30 FEG-ESEM) operated at 10 kV. EDAX and elemental mapping analysis data was also collected from the same samples at 10 kV using X-ray detector (Torr Scientific Ltd., UK) coupled with FEI/Phillips XL30 FEG-ESEM.

Evaluation of Contrast Localization—Iron Histochemistry:

Perl's Prussian Blue protocol was used to detect the presence of iron bound to the vein adventitia. Sections from the Fe-NPs labeled vein conduit were incubated with 2% aqueous potassium ferrocyanide-hydrochloric acid incubating solution (composed of equal volumes of 4% aqueous potassium ferrocyanide and 4% aqueous hydrochloric acid) for 15 minutes. The sections were washed twice for 2 minutes with ddH₂O, and then counterstained for 2 minutes with 1% Neutral Red. Subsequently, these slides were rinsed with ddH₂O (2 washes for 2 minutes each) and then dehydrated rapidly using increasing concentrations of ethanol (70%, 95%, 100%; 10 seconds each). They were then examined under polarized microscopy.

Results from the Preparation and Characterization of Fe-NP Labeled Veins

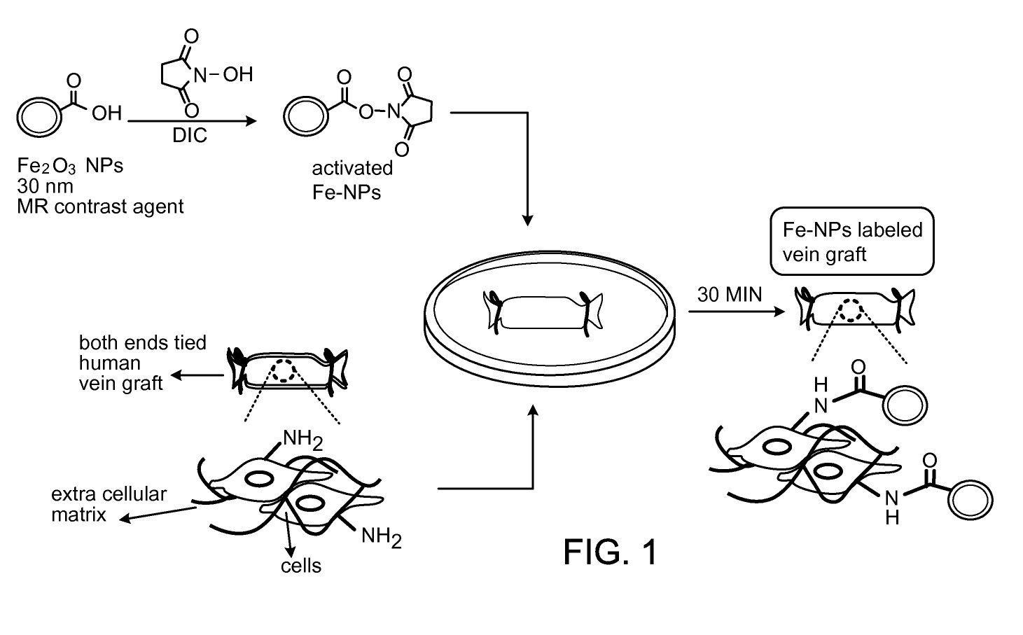

Amine groups are present in abundance on the surface of various tissues, and are available to react with N-hydroxysuccinamide. After appropriate institutional approvals, fresh discarded human vein segments from bypass surgeries and major amputations were collected and “labeled” with commercially available Fe-NPs via a two-step reaction (FIG. 1). Carboxylic groups located on the magnetic nanoparticles' surface were activated by reacting with DIC to form activated particles containing the N-hydroxysuccinamide esters. Subsequently, the activated particles were immediately used for coating the vein graft adventitia. With the luminal side protected via ligatures on either end, the test human vein sample was placed in a PBS filled Petri-dish. The activated magnetic particles (in PBS) were added and incubated for 30 minutes at room temperature. As a non-labeled control experiment, a vein segment was incubated in PBS (without Fe-NPs). Finally, all vein segments were individually and thoroughly rinsed thrice with PBS to remove unbound Fe-NPs. The current technique was applied with thirty minutes of incubation time, and this resulted in significant Fe-NP deposition on the vein.

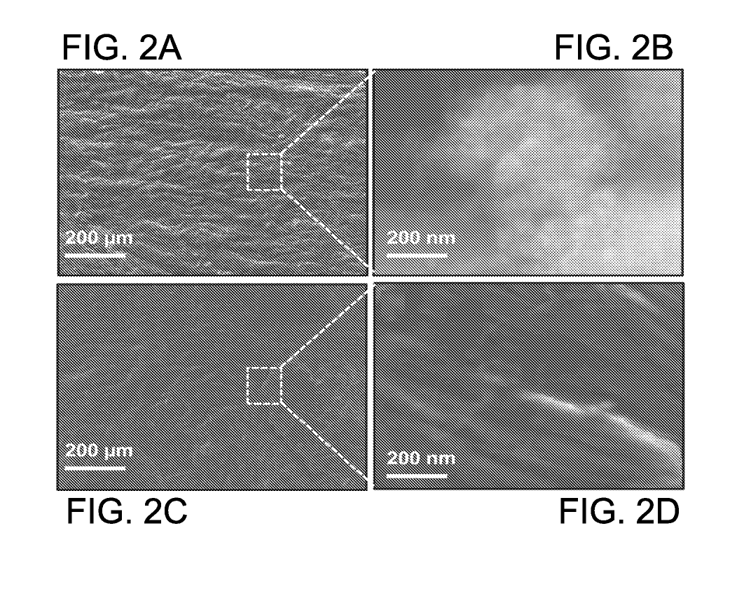

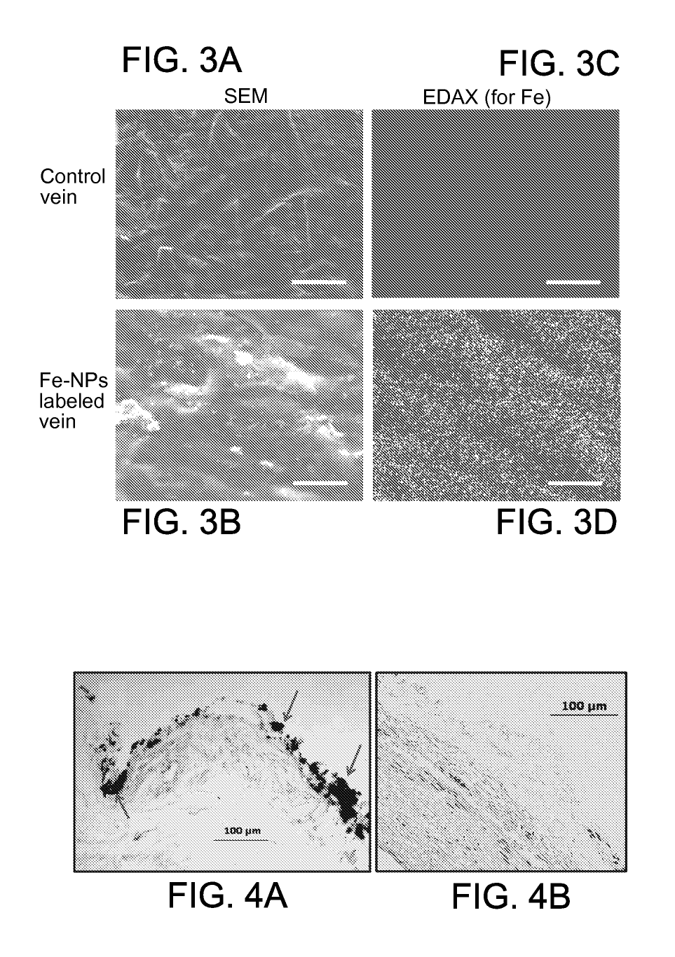

Fe-NP labeled veins were thoroughly characterized using scanning electron microscope (SEM) and energy dispersive X-ray analysis (EDAX). SEM images of labeled at 200 micrometers and at 200 nanometers (FIGS. 2A and 2B, respectively) and unlabeled veins at 200 micrometers and at 200 nanometers (FIGS. 2C and 2D, respectively) clearly showed the presence of Fe-NPs on the surface of labeled veins. Results obtained from elemental mapping analysis using SEM and EDAX (FIGS. 3A-D) showed that Fe-NP coatings covered the majority of the labeled vein surface (FIG. 3B) as opposed to the unlabeled vein surface (FIG. 3A). In addition, absence of Fe-NPs on the surface of unlabeled veins versus labeled vein surface was confirmed by EDAX (FIGS. 3C and 3D, respectively). Iron histochemistry of labeled vein cross sections (FIGS. 4A and 4B), using Perl's Prussian Blue protocol, was also performed to visualize the location of Fe-NPs on the outer surface of the vein. Dark blue/black stains in the polarized microscopy images are attributed to the presence of Fe, suggesting that Fe is confined to the vein surface (FIG. 4A). Unlabeled vein cross sections are shown in FIG. 4B.

### Experimental Details of Validation of Fe-NP Labeling by MR Imaging

MR Imaging:

T1-weighted (T1W), T2*-weighted (T2*W), and proton density-weighted (PDW) high resolution MR Imaging of labeled, control, and dose-response vein specimens was performed on a clinical 3 T MRI scanner (HDx, General Electric, Milwaukee, Wis.) equipped with 40 mT/m gradients (150 T/m/s slew rate). T1-weighted contrast images were obtained using a standard 3D spoiled gradient recalled echo sequence using the following parameters: 30° flip angle, 12.8 ms TR, 3.5 ms TE, ±32 kHz receiver bandwidth, 224×224×84 image matrix, 0.5 mm slice thickness and 6.8 cm field-of-view. T2*-weighted contrast images were obtained using the same sequence except with 5 ms TE, 100 ms TR, 160×160×68 image matrix and 4.8 cm field-of-view. Proton density-weighted images were obtained using a high sampling efficiency 3D fast spin echo (FSE) sequence with 1800 ms TR, 9 ms TE, ±16 kHz receiver bandwidth, 150×150×92 image matrix, 0.5 mm slice thickness and 4.5 cm field-of-view. Spatial resolution was 0.3×0.3×0 5 mm³ for all imaging experiments.

Results from Validation of Fe-NP Labeling by MR Imaging

Prior to EDAX and histochemistry, MR imaging was employed to validate our strategy of utilizing the magnetic properties of Fe-NPs to readily delineate the vein surface from any surrounding MR signal-generating environment. Vein segments were immersed in saline to accomplish this task. Fe-NPs create a large dipolar magnetic field gradient that acts on the water molecules that diffuse close to the particles. They have a very large transverse to longitudinal relaxitivity ratio, thus creating a predominant T2* effect resulting in MR signal hypointensity (i.e., darker), as well as generating contrast in T1-, T2-, and T2*-weighted images.

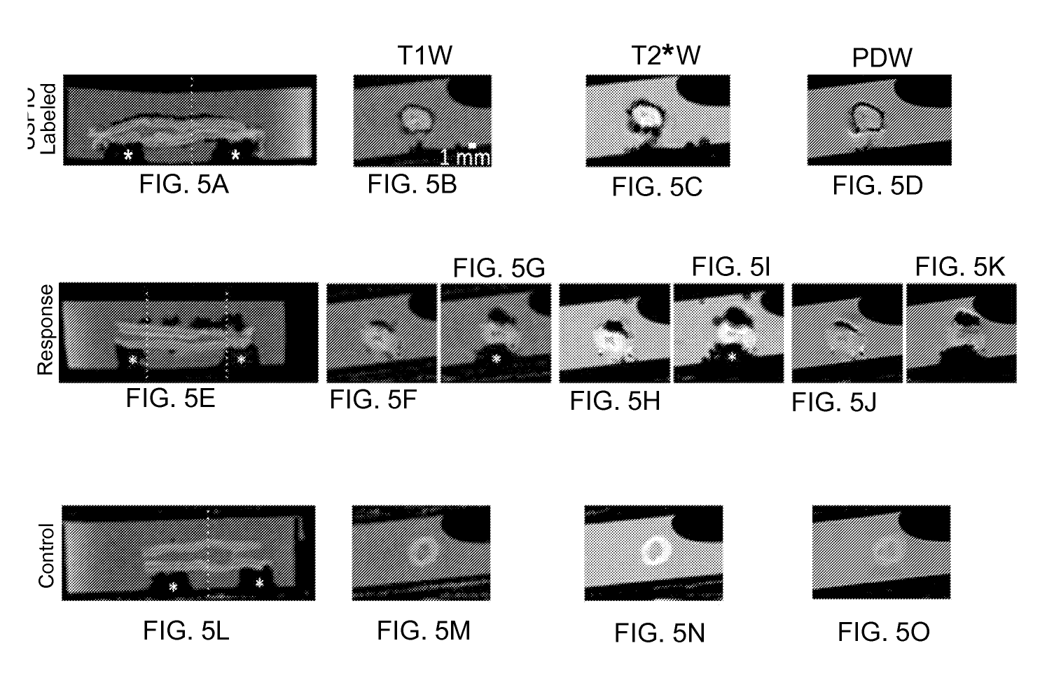

T1-weighted (T1W), T2*-weighted (T2*W), and proton density-weighted (PDW) high resolution (spatial resolution 0.3×0.3×0.5 mm³) MR Imaging of labeled and control vein specimens was performed on a clinical 3 Tesla MRI scanner (HDx, General Electric, Milwaukee, Wis.) equipped with 40 mT/m gradients (150 T/m/s slew rate) (FIGS. 5A-5O). Images are shown of three segments of the same saphenous vein: a segment circumferentially labeled with activated Fe-NPs (FIG. 5A-D); a segment with 4 equal drops of cyanoacrylate adhesive, each containing different concentration of Fe-NPs (FIG. 5E-K); the unlabeled control vein segment (FIG. 5L-O). A sagital curved multi-planar reformation of the T1W acquisition is shown in the first column; the dashed vertical lines indicate the locations of the axial images shown in the other columns. In addition, the MRI dose-response to different Fe-NPs concentrations was explored: four concentrations of Fe-NPs (50, 25, 12.5 and 6 mg) were each mixed with a drop of Cyanoacrylate adhesive and placed on the adventitia of an unlabeled vein prior to imaging. All three vein segments were imaged simultaneously (FIG. 5E-K). Additional experiments confirmed that Cyanoacrylate adhesive had no measurable effect for those image contrast weightings (not shown).

### Experimental Details of Quantitative MR Imaging

Quantitative MR Image Analysis:

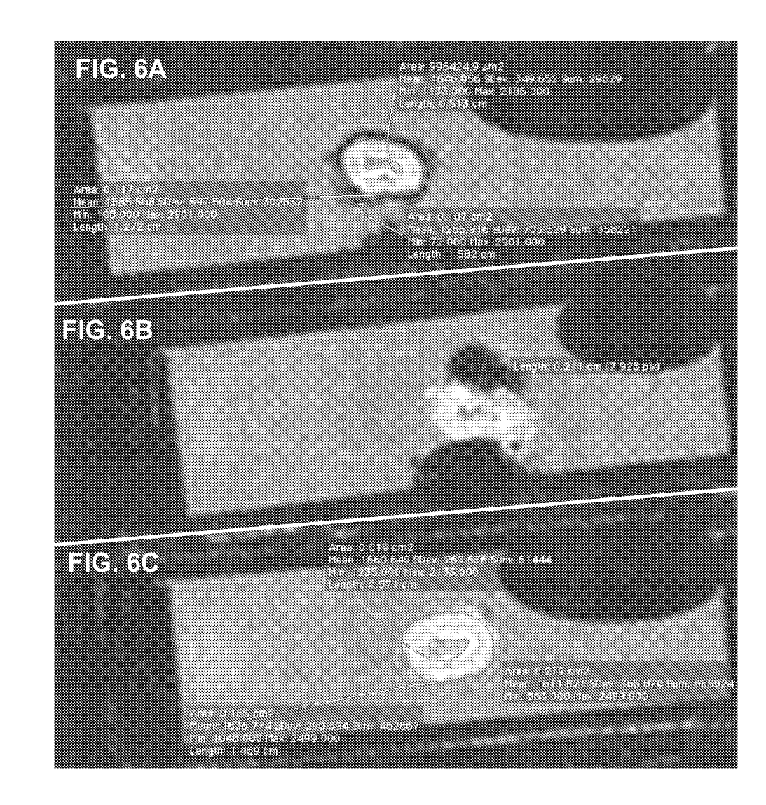

Three regions-of-interest (ROI) were drawn using direct planimetry for each of the control and labeled samples in T1W images for 17 consecutive image sections (8.5 mm longitudinal extent) sufficiently away from the Petri dish mounting setup. First, a central ROI was used to delineate the lumen. A second ROI was used to delineate the outer extent of the vessel wall tissue. Finally, the third ROI was used to delineate the region surrounding the vein. For the labeled vein, this ROI was used to delineate the outer extent of the Fe-NP susceptibility-induced signal void. For the control vein segment, this ROI was used to delineate a similar sized region containing the saline surrounding the vein (FIG. 6A-C). These ROIs were used to perform two analyses; the first analysis was aimed at testing the hypothesis that the vessel wall thickness could be accurately measured despite the presence of the Fe-NPs. For this analysis, the two inner-most ROIs (lumen and vessel wall ROIs) were assumed to represent ideal disks in order to obtain average radii as

r=\sqrt{ROI area/π}. Eq. 1

The average vessel wall thickness was then calculated by subtracting the radius corresponding to the lumen ROI from the radius corresponding to the vessel wall ROI. The unpaired t-test was used to compare the average vessel wall thickness of the labeled versus the unlabeled vein segments. This comparison was based on the assumption that the wall thickness of the two short (approximately 1.5 cm extent) vein segments did not vary significantly in thickness given that they were harvested from consecutive portions of the same donor vein.

The second analysis was aimed at assessing the ability of the Fe-NP label to effectively enhance the delineation of the vessel wall boundary in comparison to surrounding tissues. First, the average thickness of the iron oxide label was measured, as this places a limit on the image resolution required to detect it. This analysis was similar to that described for the wall thickness, except using the wall and outer region ROIs. Second, we measured the contrast-to-noise-ratio (CNR) achieved using the label. This analysis was performed by comparing the average signal in the vessel wall ROI to the average signal in the ROI surrounding the vessel wall. Average signal in each ROI was calculated by subtracting the total signal in the inner ROI from the total signal in the enclosing ROI, divided by the number of pixels in the enclosing ROI minus that in the enclosed ROI. For example, the average vessel wall signal was obtained as the total signal in the vessel wall ROI minus the total signal in the lumen ROI, divided by the number of pixels in vessel wall ROI minus the number of pixels in the lumen ROI. CNR was then computed as

$$C N R = \frac{( \begin{matrix}average vessel wall signal - \\ average surrounding signal\end{matrix} )}{{σ}_{noise}} \qquad Eq . 2$$

where σₙₒᵢₛₑ the noise standard deviation measured in an ROI placed in empty space.

A final quantitative analysis was performed for the dose-response experiment in order to determine the correlation between tissue Fe-NP concentration, and label width. For this analysis we measured the length of the susceptibility-induced signal void orthogonal to the vessel wall for each axial image containing one of the drops of the Fe-NP-loaded adhesive (FIG. 6A-C). The lengths of the signal voids were additionally measured on a single sagittal reformat of the 3D volume in order to ensure that axial slice measurements were not significantly biased by the angle of the vein segment relative to the image plane. Linear regression was used to correlate the measured widths of the Fe-NP label to the label concentration.

Results from Quantitative MR Imaging

Quantitative assessment of the ability of the Fe-NPs to enhance delineation of the vein wall from surrounding signal was performed by direct planimetry of the vessel wall and surrounding signal voids caused by the Fe-NPs using region-of-interest (ROI) measurements. In brief, three ROIs were drawn on each of the control and labeled samples in T1W images: a central ROI delineated the lumen, a second ROI delineated the outer extent of the vein-wall tissue, and a third ROI was used to delineate the region surrounding the vein. For the labeled vein, this encompassed the outer extent of the iron-oxide susceptibility-induced signal void. In the control vein segment, this region was a similarly sized ROI containing some of the surrounding saline around the control specimen (FIG. 6A-C). ROI areas were used to deduce the average thickness of the vessel wall and surrounding Fe-NP label, as well as the relative signal characteristics of the vein wall tissue and label.

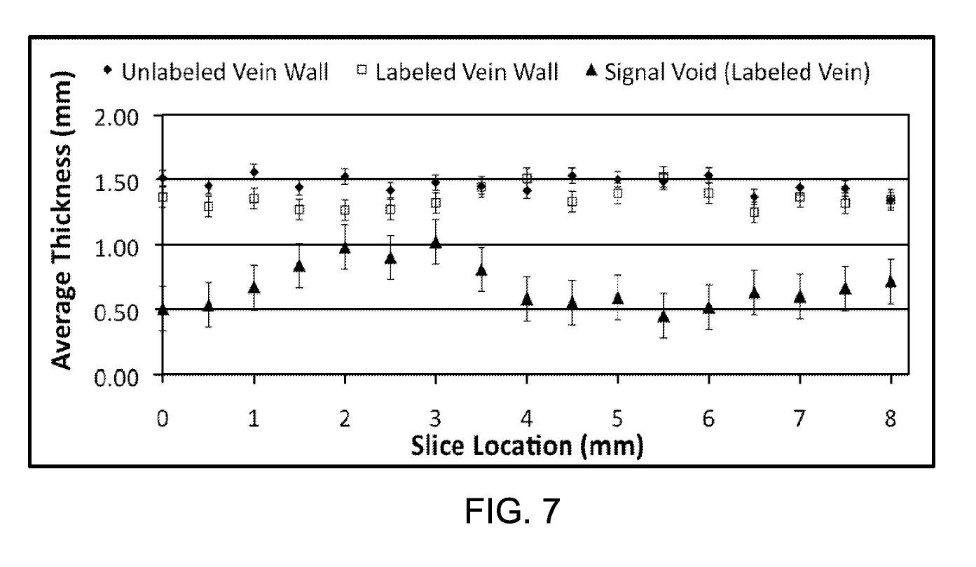

First, the hypothesis was tested that the vessel wall thickness could be accurately measured despite the presence of the iron oxide particles. The average wall thickness of the unlabeled control vein segment was 1.47±0.06 mm, while that of the labeled segment was 1.36±0.08 mm. The 0.11 mm difference in wall thickness was statistically significant (t-test, p<0.0001). There were no apparent trends in wall thickness with slice location for either vein segment (FIG. 7). Since both vein segments were obtained from consecutive portions of the same donor vein (1.5 cm length ea.), the observed difference in wall thickness was most likely due to the signal nulling effect of the Fe-NP on the labeled vein. This difference, however, is much lower than in vivo MR imaging resolutions and thus is unlikely to impact our ability to perform accurate wall thickness measurements. At present, the highest published resolution used for vein graft imaging is 0.312 mm in-plane, while most in vivo MRI is performed at resolutions higher than 0.5 mm due to scan time constraints at clinically used field strengths of 1.5 T-3 T.

A second analysis was performed to determine the ability of the Fe-NP label to enhance the delineation of the vessel wall boundary in comparison to the surrounding MR signal-generating environment. In general, this is a combination of the size of the structure to be delineated (e.g., Fe-NP label) compared to the image resolution, as well as the contrast to noise ratio of the structure (e.g., imposed between the wall tissue and the Fe-NP label).

For the ex vivo experiment, each of these two metrics was assessed separately. The average thickness of the Fe-NP label obtained from ROI measurements was 0.68±0.17 mm (FIG. 7), well within the limits of in vivo MRI resolutions. In addition, the CNR between the vessel wall and surrounding label was 20.3±2.4. Such high CNR values observed with the Fe-NP label are uncommon in vessel wall MRI; the highest CNR values encountered are near 10, and represent signal differences between the vessel wall and the signal void within the lumen in black-blood MRI. CNRs are much lower between the vessel wall and surrounding signal-generating tissues for in vivo T1W vein graft imaging. Values near zero are often encountered when the vein wall is surrounded by muscle or scar tissues. Thus, the finding of this preliminary study is that the Fe-NP label produces an effect that is global (encompassing the entire vein wall irrespective of the surrounding signal), that can be readily detected (high CNR) within the limitations of current in vivo MR imaging (extent≧to a single pixel).

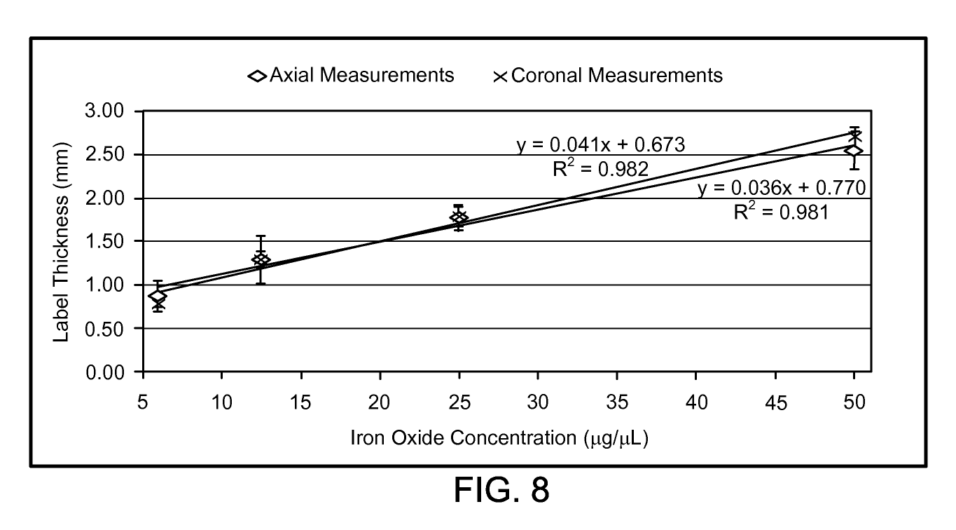

While multi-planar reformation of the vein segment with the four concentrations of Fe-NP-loaded adhesive (50, 25, 12.5 and 6 μg) demonstrated a clear dose-response of the susceptibility-induced signal void (FIG. 5A-O), a final analysis was performed to determine the correlation between Fe-NP concentration, and the thickness of the resulting label. As expected, there exist a strong linear relationship between the width of the Fe-NP susceptibility-induced signal void (FIG. 6A-C) and the iron-oxide load of the cyanoacrylate adhesive (FIG. 8). Careful control of the Fe-NP concentration will be critical since at higher concentrations (above 12.5 mg); the signal void induced by the Fe-NPs interferes with visualization of the vein wall (FIG. 5A-O). Nonetheless, the linear response of the label to Fe-NP loading suggests that this adverse effect can be minimized by optimization of the reaction conditions to employ sufficiently low Fe-NP quantities with which to label the vein wall.

An interesting effect observed in this study is underscored by the “asymmetry” of the label thickness measurements in light of the wall thickness difference between labeled and control vein segments. Specifically, while the label measured nearly 0.7 mm thick, it only encroached 0.11 mm within the vessel wall. This anisotropic effect of the Fe-NPs (namely the preferential nulling of signals outside the vessel wall rather than equally affecting the vein wall tissue signal) is likely explained by the increased mobility of the water protons in the saline compared to those in the vessel wall. As a result, the data suggests that in an in vivo environment the thickness of the label will likely be lower at the same concentration. Nonetheless, given current in vivo imaging resolutions and the large CNR attainable by the Fe-NP label developed in this work, this study suggests that sufficient label thicknesses (≧0.3 mm) can be readily achieved to delineate the label without affecting measurement of the vein wall tissue.

### Experimental Details for Covalent Immobilization of Iron Oxide Nanoparticles (Fe-NPs) on Vein Graft:

50 μL Fe-NPs were (5 mg/mL water, 30 nm size) diluted with 150 μL of double distilled water (ddH₂O). Fe-NPs have free carboxylic groups on the surface. The particle solution was centrifuged at 13,000 rpm for 6 minutes. The supernatant was removed and 200 μL of dimethyl formamide (DMF) was added, then vortexed and centrifuged for 6 minutes. The supernatant was removed and the pellet re-dispersed in 200 μL of DMF containing N,N′-diisopropylcarbodiimide (DIC, 1 mmol/L) and N-hydroxysuccinamide (1 mmol/L). The reaction continued at room temperature overnight. The activated particles were then spun down and washed with DMF (200 μL). The particles were finally re-dispersed in phosphate buffer saline (PBS) and used immediately for coating the vein graft adventitia. With the luminal side protected via ligatures on either end, the test human vein sample was distended with saline to physiologic arterial pressure (distension was done to simulate the in vivo distended vein graft and also to facilitate evaluation of the imaged vessel wall), and then placed in a PBS filled Petri-dish. The activated Fe-NPs (200 μL in PBS) were added and incubated for 30 minutes at room temperature. Finally, all vein segments were thoroughly rinsed thrice with PBS to remove nonspecifically bound nanoparticles.

The procedures described in Example 1 for characterizing Fe-NPs immobilization on vein can also be used to characterize Fe-NPs immobilization on vein graft.

### Example 2

### Immobilized Gadolinium-Based Long-Term MR Contrast Enhancement of the Vein Graft Vessel Wall

### Experimental Details of the Preparation and Characterization of Gd-DTPA Labeled Veins

Activation of Gd-DTPA for Immobilization:

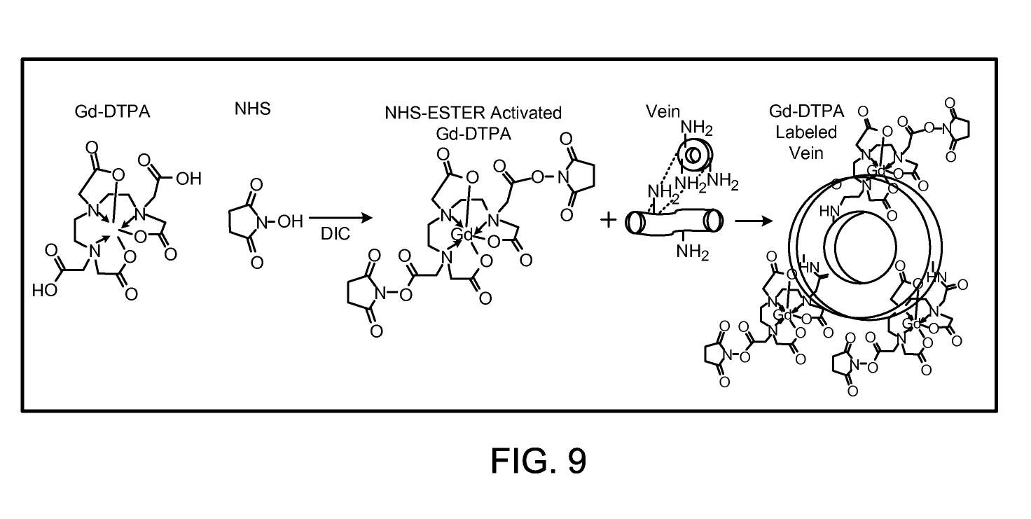

The immobilization of Gd-DTPA (Diethylenetriaminepentaacetic acid gadolinium³⁺ dihydrogen) complex on tissue was based on N-hydroxysuccinamide (NHS) ester coupling chemistry as described above for the Fe-NPs. The process of preparing gadolinium chelates for covalent binding to amines involves a reaction wherein free carboxylic acid groups in the Gd-DTPA complex are first coupled with NHS using N,N′-diisopropylcarbodiimide (DIC) (FIG. 9) by dissolving a desired concentration of Gd-DTPA in ddH₂O, adding NHS and dimethyl sulfoxide containing DIC at twice the concentration, and incubating for a period of time at room temperature. Incubation lasted either 3 hours, or, overnight under continuous stirring, as noted for individual experiments. The resulting solution contained “activated” Gd-DTPA molecules that possess NHS-ester groups (FIG. 9).

Tissue Labeling with Activated Gd-DTPA:

Veins were “labeled” with the activated Gd-DTPA by incubating for 30 minutes at room temperature in a phosphate buffered saline (PBS) solution (pH 7.4) containing the activated Gd-DTPA. Ligatures were placed on both vein ends before treatment so as to reduce endothelium exposure to the contrast agent. Following this labeling process, excess PBS was removed, and vein segments were thoroughly soaked and rinsed thrice with fresh PBS in order to remove non-bound Gd-DTPA.

Specimens:

Four fresh human saphenous vein specimens were harvested from discarded operating room tissue. Two were obtained from a below knee amputation, and two from a bypass surgery. Specimens were prepared for imaging immediately after harvesting.

Specimen Preparation:

One specimen was used to confirm the presence of gadolinium using scanning electron microscopy and elemental mapping as described below. The specimen was separated into treatment and control halves. The control half was incubated in PBS, while the treatment half was labeled in 10 mmol/L (mM) solution of the activated Gd-DTPA complex that had undergone the 3 hour preparation. To simulate in vivo black blood T1W imaging, both segments were distended to physiologic arterial pressure using corn oil and MR imaging used a fat resonance-selective saturation pulse.

Two specimens were used to obtain more direct evidence of immobilization of the activated Gd-DTPA onto vein wall tissue. One specimen was used to verify that MR image enhancement and longitudinal (R₁) and transverse (R₂) relaxation rate changes were present for tissue treated with activated, and absent for tissue treated with standard Gd-DTPA. The specimen was separated into five segments, with one control, three segments labeled in 1, 2.5, and 5 mM of activated Gd-DTPA solution prepared using the overnight protocol respectively, and one segment undergoing the same incubation process as the three labeled segments except that the solution contained 5 mM of standard Gd-DTPA (i.e., without activation by NHS ester). A second specimen was used to verify the observed relationship between tissue relaxation rates and treatment concentration. That specimen was separated into six segments, with one used as a control and the remaining five labeled in 1, 2, 4, 6, and 10 mM of activated Gd-DTPA solution prepared using the overnight protocol, respectively.

Finally, one specimen was used to study the long-term stability of the immobilized Gd-DTPA. The specimen was separated into two segments, with one half used as a control, and the other half labeled in 10 mM solution of activated Gd-DTPA prepared using the 3 hour protocol. Sodium azide (a preservative that prevents tissue spoilage, was subsequently added to the saline used in the imaging setup (see below), and the specimen was incubated in a shaker over four months at 37° C. to simulate in vivo conditions. R₁ and R₂ rates were measured at 0 and 122 days following harvest. The R₂ rate was additionally measured at 22 days.

Specimen Preparation for Imaging:

To avoid collapse of the thin-walled veins for imaging and to simulate the in vivo signal environment, the ends of vein segments were surgically tied and either saline or corn oil as pertaining to each experiment was used to distend them to 80 mmHg. Distended veins were then suspended in saline-filled Petri dishes with the use of two 2.5 mm polystyrene struts. Prepared Petri dishes were sealed using surgical tape. With the exception of the long-term stability experiment, specimens were stored at 4° C. for no longer than 12 hours between preparation and imaging. To avoid cross-contamination, each vein segment for each experiment was handled with separate surgical instruments and mounted in a separate Petri dish.

MRI Equipment:

All experiments were performed on a 3 T GE HDx MR imager (General Electric, Milwaukee, Wis.), equipped with 40 mT/m, 150 T/m/s gradients. A wrist extremity bird-cage transmit/receive coil was used for radio-frequency (RF) excitation and signal reception.

MR Imaging:

MR image enhancement was assessed in 3D spoiled gradient recalled echo (SPGR) T1W images (4 ms TE, 30 ms TR, 40° FA, ±32 kHz bandwidth). The signal-to-noise ratio was measured for each vein segment as

SNR=Sₜᵢₛₛᵤₑ/σ_{image} Eq. 3

where Sₜᵢₛₛᵤₑ the mean signal intensity in a region-of-interest (ROI) containing vein wall tissue, and where the standard deviation of noise in the image was calculated as

σ_{image}=Sₙₒᵢₛₑ/\sqrt{π/2} Eq. 4

with Sₙₒᵢₛₑ the mean signal intensity in a ROI containing air. SNR measurements are reported as the mean and standard deviation (SD) over multiple ROIs for each vein segment.

ESEM-EDAX Imaging:

An environmental scanning electron microscope (ESEM; FEI/Philips XL30 FEG-ESEM, FEI, Hillsboro, Oreg.) equipped with an energy dispersive X-ray (EDAX) spectrometry module (Torr Scientific Ltd., UK) was used to obtain micrographs of 4 μm-thick sections of one specimen at 10 kV. Elemental mapping analysis to identify the presence of Gd was performed using the EDAX technique in which characteristic X-rays emitted from the sample after electron bombardment are used to identify element-specific signatures. Combined with ESEM, spectra analyzed in a raster fashion produce a two dimensional image of the spatial distribution of gadolinium.

Quantitative MR Relaxation Rate Measurements:

For the long-term stability specimen, R₂ was measured using a multi-echo 2D Carr-Purcell-Meiboom-Gill (CPMG) sequence. A 3D CPMG sequence with composite (90ₓ-180_{y}-90ₓ) refocusing pulses was used to measure the R₂ of specimens with multiple labeling concentrations. The increased SNR and reduced indirect and stimulated echoes at longer echo train lengths afford the latter sequence enhanced accuracy for graded R₂ comparisons. For 2D CPMG, images were acquired at eight echo times (11 ms echo spacing, 1500 ms TR). For 3D CPMG, images were produced at 16 echo times (11.6 ms echo spacing, 1600 ms TR).

R₁ was measured using a high sampling efficiency 3D FSE (8 ETL, 7 ms echo spacing) with an 8.64 ms adiabatic inversion recovery (IR) preparatory pulse. For the long-term stability experiment, images were obtained at five inversion times (TI) ranging from 50 milliseconds to 1000 milliseconds at 250 milliseconds intervals, with a TR of 1800 milliseconds. For the two experiments with multiple concentrations, images were obtained at 11 TIs, with 8 TIs between 125 and 1000 ms at 125 ms intervals and 3 additional TIs at 50, 1250, and 1500 ms, with a TR of 3000 milliseconds.

Relaxation rates were obtained by fitting signal intensities in ROIs using an idealized signal model; R₂ rates were obtained by fitting to the model

s(t)=αe^{-tR}^{₂} Eq. 5

where t the echo times, and α a constant reflecting the spin density. R₁ rates were obtained by fitting to the model

s(t)=α(1−(1−k)e^{-t·R}^{₁}−ke^{-TR·R}^{ᵢ}) Eq. 6

where t the inversion times, α as before, and −1≦k≦1 a constant accounting for the efficiency of the inversion pulse. Fitting was performed with a non-linear optimization routine (Matlab R2008b, Mathworks, Natick, Mass.) employing a non-negativity constraint for each fitted variable. Mean relaxation rates are reported by averaging fits from no less than twenty ROIs per vein segment.

Statistical Analysis:

For the experiment with segments incubated in both activated and non-activated Gd-DTPA, one-way analysis of variance (ANOVA) of the R₁ and R₂ rates was used to test whether segments labeled with activated Gd-DTPA had significantly different relaxation rates than the control segment and the segment incubated in standard Gd-DTPA. A p value less than 0.05 was considered significant, indicating a significantly different mean relaxation rate compared to the control due to the presence of Gd-DTPA.

For the two experiments with multiple concentrations of activated Gd-DTPA, linear regression was used to determine the relationship between the treatment agent concentration and tissue R₁ and R₂ rates, and to subsequently determine the ratio of r₂ to r₁ relaxivity of the agent. Specifically, the “pseudo-relaxivities” rᵢ′, i=1, 2, of the agent was first defined as the regression coefficients of the models

Rᵢ=Rᵢ⁰+rᵢ′×Cₜᵣₑₐₜₘₑₙₜ, i=1,2, Eq. 7

where Rᵢ⁰ is the tissue relaxation rate in the absence of contrast agent, and Cₜᵣₑₐₜₘₑₙₜ the treatment concentration of activated Gd-DTPA. A Pearson correlation coefficient r≧0.9 was considered indicative of no saturation of the available binding sites on the tissue having occurred for the particular reaction conditions. In this event, one may assume that the treatment concentration is directly proportional to the resulting tissue concentration, up to a constant factor related to each particular specimen's uptake rate for the incubation conditions. That is, it can be inferred that the relationship Cₜᵢₛₛᵤₑ=αCₜᵣₑₐₜₘₑₙₜ holds for some constant α specific to each individual experiment. Consequently, the ratio of r₂ to r₁ relaxivity of the implanted agent can be computed from the observed pseudo-relaxivities, rᵢ′, since the constant drops out.

For the long term stability experiment, tests were conducted to determine whether relaxation rates differed between labeled and control segments at each individual time point using the unpaired two-tailed student t-test. A p value less than 0.05 was considered significant, indicating a significantly different mean relaxation rate for the labeled versus control segments of the veins due to the presence of Gd-DTPA. Second, tests were conducted to determine whether the relaxation rate of each segment differed between time points. ANOVA was used to perform this comparison across the three time points where R₂ was measured, and the unpaired two-tailed student t-test was used for the equivalent comparison of R₁ rates, as those were only measured at two time points. In either case, a p value greater than 0.05 was considered indicative of no significant change in the mean relaxation rate of the particular vein segment over the experiment duration.

Results from the GD-DTPA Labeled Vein

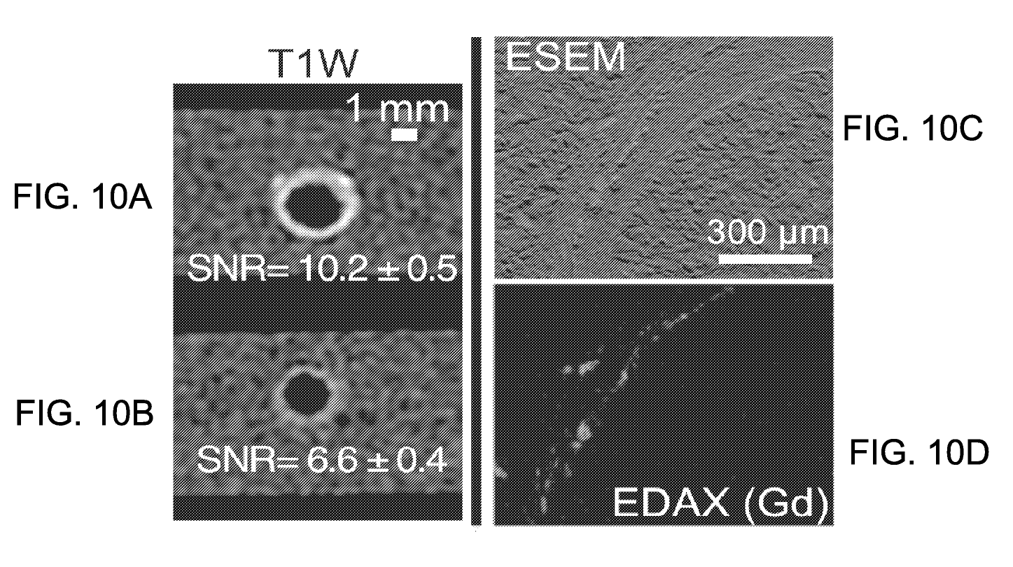

Labeled vein segments presented significantly higher T1W signal intensity than controls (FIGS. 10A-D, 11A-K, and 12A-B), with SNR between 1.5 and 4.5 times higher for labeled than for control vein segments. EDAX confirmed the presence of gadolinium (Gd) on the labeled vein segment only (FIG. 10A) versus unlabeled vein segment (FIG. 10B), concentrated primarily on the outer surface of the adventitia. ESEM micrograph (FIG. 10C) and EDAX elemental mapping (FIG. 10D) of the labeled vein confirmed the presence of Gd. However, Gd ions were observed diffusely throughout the vessel wall, in agreement with all MR image results that showed increased signal intensity throughout the wall (FIGS. 10A-D, 11A-K, and 12A-B). The signal intensity of surrounding saline did not differ between the labeled and control segments in any experiments, indicating absence of free Gd-DTPA at the time of imaging.

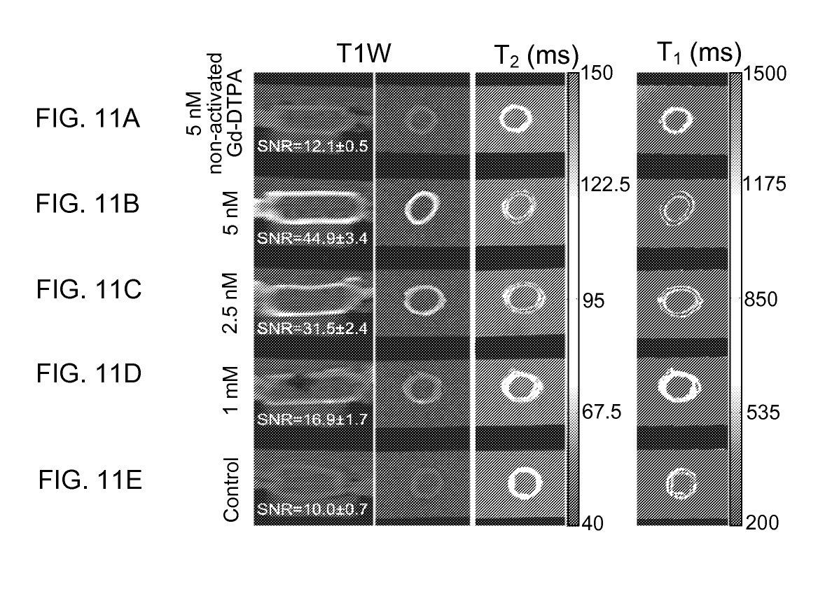

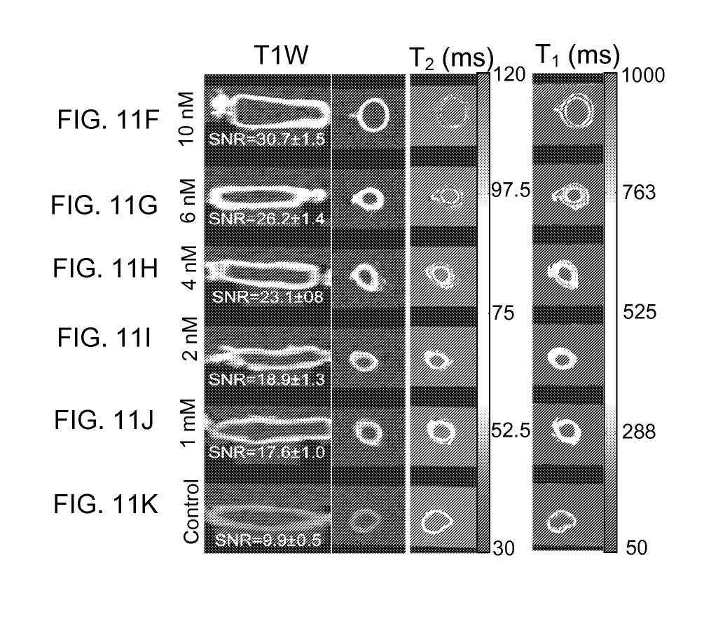

The increased T1W signal and positive EDAX finding offered preliminary confirmation that the activated Gd-DTPA complex could selectively enhance the relaxation properties of the target tissue. Evidence supporting immobilization onto tissue constituents followed more directly from the experiments portrayed in FIGS. 11A-K and 12A-B. Specifically, the R₁ and R₂ rates of a vein incubated in 5 mM of non-activated Gd-DTPA (FIG. 11A) were no different than those of the control (FIG. 11E) (R₂: 10.5±0.8 s⁻¹ treated versus 10.6±1.0 s⁻¹ control, p=0.561; R₁: 0.89±0.03 s⁻¹ treated versus 0.87±0.07 s⁻¹ control, p=0.102), confirming that vein wall tissue did not retain any significant amount of non-activated Gd-DTPA. In contrast, all segments of that vein incubated in activated Gd-DTPA presented significantly higher relaxation rates (R₂: 13.7±0.8 s⁻¹, 17.8±1.5 s⁻¹, and 24.1±2.4 s⁻¹ for 1, 2.5, and 5 mM respectively (FIG. 11D, C, B, respectively), p<0.001 versus control; R₁: 1.29±0.16 s⁻¹, 2.12±0.26 s⁻¹, and 3.58±0.28 s⁻¹ for 1, 2.5, and 5 mM respectively, p<0.001 versus control), confirming the presence of gadolinium. This strongly suggested successful implantation of only activated Gd-DTPA onto the vein wall tissue, and clarified that in the absence of NHS-ester activation, surface interactions between Gd-DTPA and the vein wall tissue are limited.

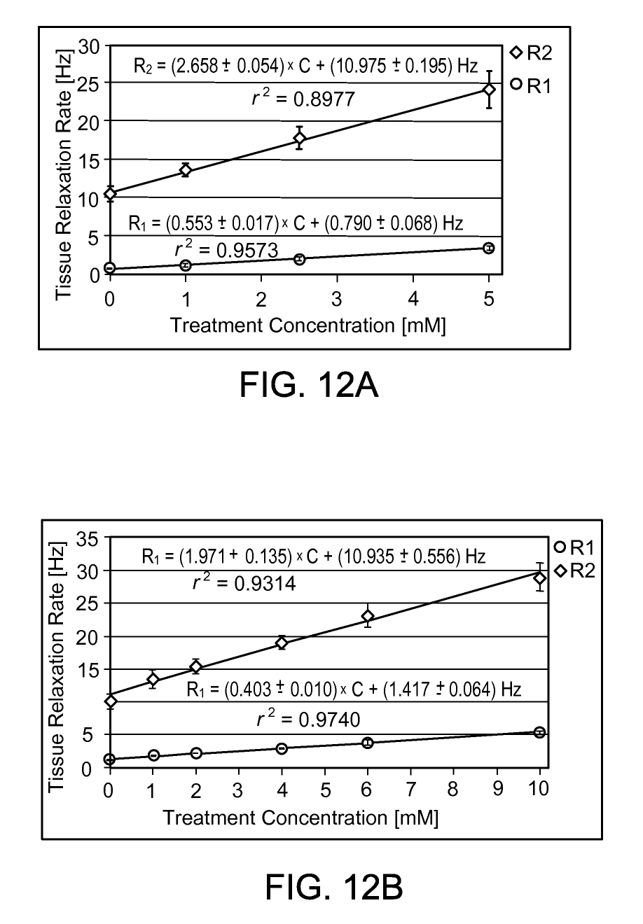

Additional information regarding the particular nature of the interaction between activated Gd-DTPA and vein tissue was obtained from the rate of change of the relaxation rates with respect to the concentration of the implanted agent. The observed relationship between relaxation rate and incubation concentration was highly linear for both R_{1,2} (Pearson r>0.95 for all fits, FIGS. 12A and 12B) over the tested range of 0-10 mM. As a result, the number of binding sites on the vein tissue was not saturated, and that the tissue concentrations achieved were directly proportional to the treatment concentration, up to a multiplicative factor specific to each particular specimen. Using the pseudo-relativities (slopes of the fitted models) under this linearity assumption, the ratio of r₂ to r₁ relaxivity of the activated Gd-DTPA was calculated to be 4.81±0.18 and 4.89±0.36 (FIGS. 12A and 12B, respectively) in these two experiments.

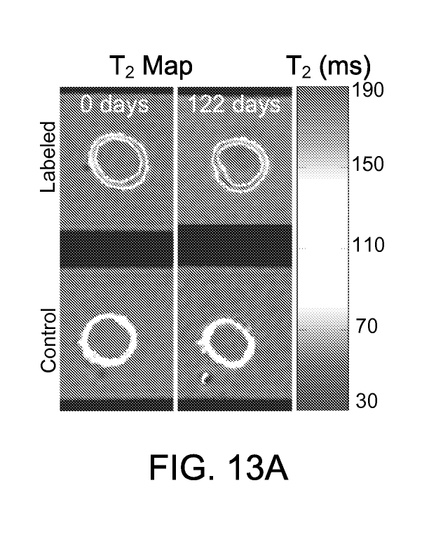

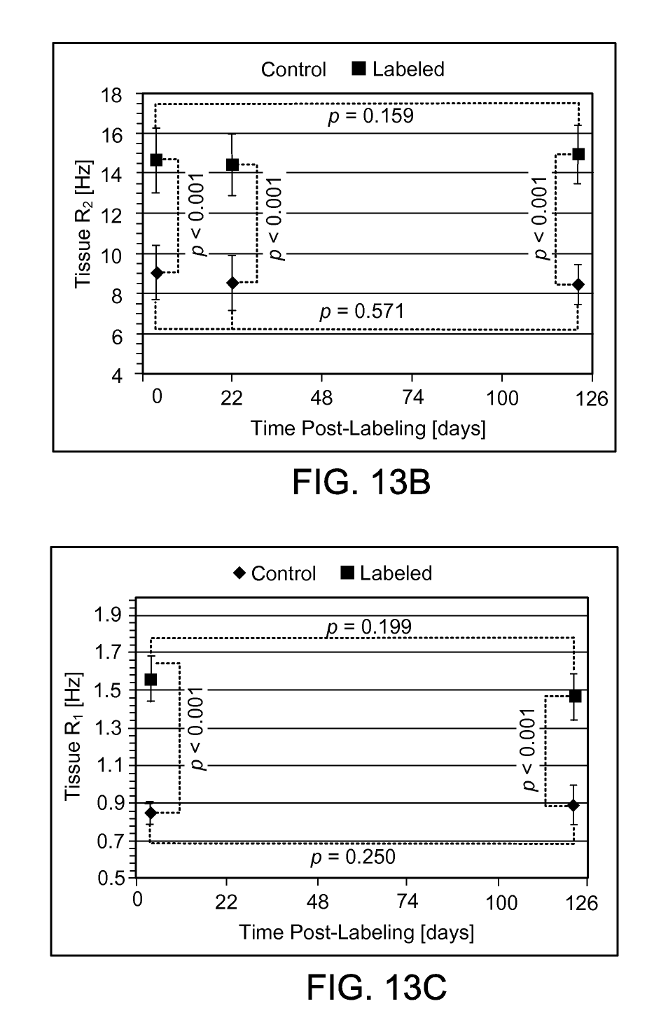

Additionally, the cross-linkage of Gd-DTPA to vein wall tissue was stable over a four-month period; both R₁ and R₂ rates remained stable throughout this period for both the control (p>0.250, FIG. 13A) and the labeled (p>0.159, FIG. 13A) vein segments. Labeled segment R₂ ranged from 14.66±1.61 s⁻¹ at harvest to 14.96±1.43 s⁻¹ at 122 days, and was significantly higher than that of the control (9.06±1.35 s⁻¹ at harvest to 8.45±1.00 s⁻¹ at 122 days) at all three time points (p<0.001, FIG. 13B). R₁ was also significantly different between the control and labeled segments at both time points (p<0.001, FIG. 13C); the labeled segment's R₁ was 1.56±0.12 s⁻¹ at harvest and 1.52±0.09 s⁻¹ at 122 days, compared to 0.86±0.05 s⁻¹ at harvest and 0.89±0.11 s⁻¹ at 122 days for the control.

The results described above show that immobilized contrast agents such as the Gd-DTPA is a valid proposition that can be realized for significant gains. The 1.5-4.5-fold increase in T1W SNR observed in vitro was readily traded for a 2- to 20-fold increase in T1W imaging speed while maintaining the SNR, spatial resolution, and CNR expected for unenhanced tissue. For lower extremity vein bypass grafts, where the full vessel wall can be delineated in T1W images, a 4-fold reduction in imaging time that can be realized by a 2-fold increase in SNR—well within the SNR increases demonstrated here—would immediately enable us to extend in vivo surveillance to the entire graft in the same scan time as we currently use for only a short segment.

Additionally, the results described above demonstrate that these immobilized contrast agents a) are specific to the tissue they are implanted on, b) they possess a robust relationship with labeling treatment conditions, and c) are maintained over a significant period of time. Regarding the contrast agent's tissue specificity, these results demonstrate that the implantable Gd-DTPA attached to the target tissue, as opposed to being temporarily absorbed. Only veins treated with activated Gd-DTPA presented increased relaxation rates compared to control, while a segment treated with standard (unactivated) Gd-DTPA under otherwise identical conditions presented no significant differences as evidenced by the r₂ relaxivity which was nearly 5 times larger than its r₁ relaxivity (FIG. 12A-B). Preferential R₂ enhancement is characteristic of increased low-frequency fluctuations associated with Gd ions attached onto large molecular weight compounds. According to standard Bloembergen, Pound and Purcell theory, longitudinal relaxation rates are most sensitive to random magnetic field fluctuations at, and near, the Larmor frequency and twice the Larmor frequency, while transverse relaxation rates are additionally sensitive to very low (DC) frequency fluctuations. A dramatic reduction in tumbling frequency, as achieved by bonding Gd chelates to e.g., human albumin for MS-325, and as perhaps afforded by anchoring Gd-DTPA to solid vein wall tissue constituents in this study, preferentially enhances transverse over longitudinal relaxation as only the latter is sensitive to very low frequency magnetic fluctuations.

The relationship between treatment conditions and signal enhancement was complex. It was affected by the agent tissue concentration achievable, tissue composition, as well as the relaxivities of the agent, which are in turn affected by the cross-linkage. The tissue concentration that can be successfully implanted depends not only on the concentration of agent used in the labeling reaction, but also on the number of available binding sites (free primary amines) on the exposed tissue surfaces. The former appears to have been a limitation in some experiments; the 3 hour Gd-DTPA activation protocol resulted in significantly smaller differences in tissue R_{1,2} at the same treatment concentration as compared to the overnight preparation. It thus appears that the reaction yielded a significantly larger percentage of activated Gd-DTPA under constant stirring and extended duration.

The availability of binding sites may still pose a limitation, since it may vary significantly depending on the type and condition of tissue. The increased r₂ to r₁ relaxivity ratio, characteristic of immobilized gadolinium chelates and observed here, may exacerbate this since higher tissue concentrations, necessary to achieve further signal enhancement from T₁ effects, will ultimately be limited by the accompanying T₂ shortening. For the saphenous veins examined, binding sites were not saturated yet the achieved tissue concentrations sufficed to impart large signal changes. The right-hand panel of FIGS. 11A-K (FIGS. 11F-K) indicated a T₁ change from 763.3±43.5 ms (control) to 185.7±7.9 ms (10 mM treatment), accompanied by a T₂ change from 99.8±11.2 ms to 34.7±2.7 milliseconds. However, the resulting 3.1-fold SNR increase of that vein in a standard T1W acquisition equates to a 9-fold scan time reduction that can be readily realized using standard methods such as signal averaging reduction and parallel imaging. Overall signal increases of the order of 4.5-fold achieved in this study should suffice for most conceivable desired benefits for state-of-the-art vessel wall imaging. Nonetheless, should saturation of binding sites prove a limitation for other applications, dendrimers with multiple Gd ions per molecule may be considered, since the simple cross-linking chemistry we used is readily portable to many molecules.

Regarding the maintenance of the immobilized contrast agent, results demonstrated the in vitro durability of the Gd-DTPA cross-linkage in a vein incubated for four months (FIGS. 13A-C). Relaxation rate differences between a treated and an untreated vein segment stably persisted throughout the duration of the experiment, indicating reasonably long-term binding of the agent to tissue constituent.

### Experimental Details for Covalent Immobilization of Gd-DTPA on Vein Graft

Diethylenetriaminepentaacetic acid gadolinium³⁺ dihydrogen (Gd-DTPA, 1 equivalent) was added to the 2 mL of dimethyl sulfoxide (DMSO) in a glass vial, to that N,N′-Diisopropyl carbodiimide (DIC, 10 equivalents) was added and placed on shaker. After 1 hour appropriate amount of N-hydroxy succinamide (NHS, 10 equivalents) was added to the vial and shaking was continued at room temperature. After 12 hours, with the luminal side protected via ligatures on either end, the human vein was distended with saline to physiologic arterial pressure (distension was done to simulate the in vivo distended vein graft and also to facilitate evaluation of the imaged vessel wall), and was placed in vial (which contain DMSO solution of Gd-DTPA), then after two minutes 8 mL of double distilled was added, then vial was left at room temperature for 30-45 minutes. After incubation, vein was removed from the vial and placed in 25 mL of phosphate buffer saline (PBS). After gentle shaking of vial for 2 minutes with hand, PBS has removed and replenished with fresh PBS. Similar washing steps were repeated four times to remove non-covalently bound Gd-DTPA. After final wash vein was removed and placed in fresh PBS.

The procedures described in Example 2 for characterizing Gd-DTPA labeled vein can also be used to characterize Gd-DTPA labeled vein graft.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-2. (canceled)

3. A method of labeling a tissue or organ ex vivo or in vivo, the method comprising contacting the tissue or organ with a contrast agent capable of binding to the tissue or organ of interest, under conditions sufficient for the contrast agent to be immobilized on the tissue or organ.

4. The composition of claim 3, wherein the organ of interest is part of the circulatory system.

5. The composition of claim 3, wherein the contrast agent is an X-ray contrast agent, computed tomography (CT) contrast agent, single photon emission computed tomography (SPECT) contrast agent, positron emission tomography (PET) contrast agent, infrared contrast agent, magnetic resonance imaging (MRI) contrast agent, or fluorescent dye.

6. The composition of claim 3, wherein the contrast agent comprises a metal chelator that chelates a detectable metal atom.

7. The composition of claim 3, wherein the contrast agent is paramagnetic or radioactive.

8. The composition of claim 3, wherein the contrast agent is a paramagnetic particle or ultra-small paramagnetic particle.

9. The composition of claim 3, wherein the contrast agent is immobilized on the tissue or organ via a covalent or non-covalent bond.

10. The method of claim 3, wherein the method is carried out during, or as part of, a surgical procedure.

11. The composition of claim 3, wherein the tissue or organ comprises a functional group that is selected from the group consisting of an amino group, a thiol group, or a hydroxyl group.

12. The composition of claim 3, wherein the contrast agent comprises a functional group that is suitable for reacting with a functional group present on the tissue or organ.

13.-16. (canceled)
